(19) United States
(12) Patent Application Publication
(10) Pub. No.: US 2024/0400989 A1
(43) Pub. Date: Dec. 5, 2024

(54) METHOD FOR PROMOTING PROLIFERATION OF IMMUNE CELLS

(71) Applicant: ORICELL THERAPEUTICS CO., LTD., Shanghai (CN)

(72) Inventors: Hao GUO, Shanghai (CN); Ruirong YI, Shanghai (CN); Yanni ZHANG, Shanghai (CN); Siye CHEN, Shanghai (CN); Xiaowen HE, Shanghai (CN)

(73) Assignee: ORICELL THERAPEUTICS CO., LTD., Shanghai (CN)

(21) Appl. No.: 18/652,232

(22) Filed: May 1, 2024

Related U.S. Application Data
(63) Continuation-in-part of application No. 17/040,865, filed on Sep. 23, 2020, now Pat. No. 12,005,080.

(30) Foreign Application Priority Data
Mar. 26, 2018 (CN) 201810251875.7

(51) Int. Cl. C12N 5/0783; C07K 14/775

(52) U.S. Cl. CPC C12N 5/0636; C07K 14/775; C12N 2510/00

(57) ABSTRACT

Disclosed is a method for promoting immune cell proliferation. The method comprises the following step: upregulating the expression of low-density lipoprotein receptor-related proteins or fragments thereof in immune cells.

### CROSS-REFERENCE TO RELATED APPLICATIONS

This application is a Continuation-in-Part of U.S. application Ser. No. 17/040,865, filed Sep. 23, 2020, now allowed; which is a 371 of PCT/CN2019/079576, filed Mar. 25, 2019. Priority to Chinese patent application 201810251875.5, filed Mar. 26, 2018, is claimed. All of the above applications are incorporated herein by reference in their entireties.

### REFERENCE TO A SEQUENCE LISTING

In accordance with 37 CFR § 1.833-1835 and 37 CFR § 1.77 (b) (5), the specification makes reference to a Sequence Listing submitted electronically as a xml file named “552695US_ST26a.xml”. The xml file was generated on Apr. 30, 2024 and is 130,640 bytes in size. The entire contents of the Sequence Listing are hereby incorporated by reference.

### FIELD

The present application relates to a method for promoting the proliferation of an immune cell. In particular, the method of the present application can up-regulate the expression of a low density lipoprotein receptor-associated protein or fragment thereof in the immune cell.

### BACKGROUND

Chimeric Antigen Receptor T-Cell Immunotherapy (CAR-T) involves modifying a T cell by means of genetic engineering to allow it to express a Chimeric antigen receptor (CAR) and recognize an antibody of a tumor cell surface antigen, thereby enhancing a specific killing of tumor by the T cell. CAR generally comprises specially recognizing a Single chain variable fragment (scFv), a hinge region, a transmembrane region, and an intracellular signal transduction region of a Tumor associated antigen (TAA).

Recently, CAR-T immunotherapy has significant effects in the treatment of acute leukemias and non-Hodgkin's lymphomas, but the CAR-expressing immune cells have an extremely limited proliferation ability.

### SUMMARY

The present application provides a genetically modified immune cell, and a method for promoting the proliferation of immune cells. The method provided in the present application can up-regulate the expression of the low density lipoprotein receptor-associated protein or fragment thereof in the immune cell. The method provided in the present application can further promote the production of a memory immune cell. The method provided in the present application can further inhibit the differentiation of immune cells. The method provided in the present application can further enhance the release of a cytokine from immune cell. The method provided in the present application can further enhance the ability of the immune cells to kill a tumor. Moreover, the method provided in the present application can be used to prevent the recurrence of tumors in a subject. The present application further provides a method for treating tumors in a subject in need thereof. The present application further provides a composition including the genetically modified immune cell, as well as use of the genetically modified immune cell and the composition in the manufacture of a drug. The present application further provides a method for producing the genetically modified immune cells.

In one aspect, the present application provides a method for promoting the proliferation of immune cells, including a following step: upregulating the expression of the low density lipoprotein receptor-associated protein or fragment thereof in the immune cell.

In one aspect, the present application provides a method for promoting the production of memory immune cells, including a following step: upregulating the expression of the low density lipoprotein receptor-associated protein or fragment thereof in the immune cells, thereby promoting the differentiation of the immune cells to the memory immune cells.

In one aspect, the present application provides a method for inhibiting the differentiation of immune cells, including a following step of upregulating the expression of the low density lipoprotein receptor-associated protein or fragment thereof in the immune cells, thereby inhibiting the differentiation of the immune cells to the differentiated immune cells.

In one aspect, the present application provides a method for enhancing the release of cytokines from immune cells, including a following step: upregulating the expression of the low density lipoprotein receptor-associated protein or fragment thereof in the immune cell.

In certain embodiments, the cytokines comprise interleukin, interferon and/or tumor necrosis factor. In certain embodiments, the cytokines comprise IL-2, IL4, IL6, IL7, IL10, IL12, TNF-α and/or IFNγ.

In one aspect, the present application provides a method for enhancing the ability of immune cells to kill tumors, including a following step: upregulating the expression of the low density lipoprotein receptor-associated protein or fragment thereof in the immune cells.

In one aspect, the present application provides a method for preventing the tumor recurrence in a subject, including administering immune cells to a subject susceptible to a tumor. wherein the expression of the low density lipoprotein receptor-associated protein or fragment thereof is upregulated in the immune cells.

In one aspect, the present application provides a method for treating tumors in a subject in need thereof, including a following step: administering immune cells to the subject, wherein the expression of the low density lipoprotein receptor-associated protein or fragment thereof is upregulated in the immune cell.

In certain embodiments, the tumor is selected from the group consisting of liver cancer, lung cancer, leukemia, and mesothelioma.

In certain embodiments, the method comprises an in vivo method and an in vitro method.

In certain embodiments, the immune cell comprises a lymphocyte. In certain embodiments, the immune cell comprises a T cell. In certain embodiments, the T cell comprises a memory stem cell-like T cells (TSCM) and/or a central memory T cells (TCM). In certain embodiments, the TSCM is CCR7⁺ and/or CD62L⁺. In certain embodiments, the TSCM further has one or more properties selected from the group consisting of CD45RA⁺ or CD45RA⁻; CD45RO⁺ or CD45RO⁻, CD27⁺, CD28⁺, CD127⁺, CD122⁺, CD3⁺, CD4⁺, and CD8⁺.

In certain embodiments, the immune cell comprises genetically modified immune cells, and the genetically modified immune cells express chimeric antigen receptor (CAR) or a T cell receptor (TCR). In certain embodiments, the genetically modified immune cell comprises a genetically modified T cell, NK cell, B cell, dendritic cell, or macrophage.

In certain embodiments, the CAR comprises an intracellular domain, and the intracellular domain comprises a signal transduction domain and/or a costimulatory domain.

In certain embodiments, the signal transduction domain comprises a moiety selected from the group consisting of a signal transduction domain of CD3ζ, a signal transduction domain of CD3δ, and a signal transduction domain of CD3ε. In certain embodiments, the signal transduction domain comprises an amino acid sequence as set forth in SEQ ID NO: 18 or an amino acid sequence having at least 80% homology thereof. In certain embodiments, a nucleic acid molecule encoding the signal transduction domain comprises a nucleic acid sequence as set forth in SEQ ID NO:17 or a nucleic acid sequence having at least 80% homology thereof.

In certain embodiments, the costimulatory domain comprises a moiety selected from the group consisting of a costimulatory domain of CD27, a costimulatory domain of CD28, and a costimulatory domain of 4-1BB. In certain embodiments, the costimulatory domain comprises an amino acid sequence as set forth in any one of SEQ ID NO:14, and SEQ ID NO:16 or an amino acid sequence having at least 80% homology thereof. In certain embodiments, a nucleic acid molecule encoding the costimulatory domain comprises a nucleic acid sequence as set forth in any one of SEQ ID NO:13, and SEQ ID NO:15 or a nucleic acid sequence having at least 80% homology thereof.

In certain embodiments, the CAR comprises a hinge region. In certain embodiments, the hinge region comprises a moiety selected from the group consisting of a hinge region of IgG4, a hinge region of IgG1, and a hinge region of CD8. In certain embodiments, the hinge region comprises an amino acid sequence as set forth in SEQ ID NO: 31. In certain embodiments, a nucleic acid molecule encoding the hinge region comprises a nucleotide sequence as set forth in SEQ ID NO: 32.

In certain embodiments, the CAR comprises a transmembrane region. In certain embodiments, the transmembrane region comprises a moiety selected from the group consisting of a transmembrane region of CD8, a transmembrane region of CD28, and a transmembrane region of CD24. In certain embodiments, the transmembrane region comprises an amino acid sequence as set forth in SEQ ID NO: 33. In certain embodiments, a nucleic acid molecule encoding the transmembrane region comprises a nucleotide sequence as set forth in SEQ ID NO: 34.

In certain embodiments, the CAR comprises a targeting moiety. In certain embodiments. the targeting moiety comprises ScFv.

In certain embodiments, the targeting moiety specially binds to and/or recognizes tumor antigen. In certain embodiments, the targeting moiety specially binds to and/or recognizes a target selected from the group consisting of B lymphocyte surface antigens, TNF family members, HER family members, and GPC family members. In certain embodiments, the targeting moiety specially binds to and/or recognizes a target selected from the group consisting of CD19, BCMA, HER2, Mesothelin, and GPC3,

In certain embodiments, the targeting moiety is an antibody or antigen-binding fragment thereof that specially binds to and/or recognizes CD19, and the antibody or antigen-binding fragment thereof comprises a light chain variable region including LCDR1-LCDR3, wherein the LCDR1 comprises an amino acid sequence as set forth in SEQ ID NO: 46 or an amino acid sequence having at least 80% homology thereof; the LCDR2 comprises an amino acid sequence as set forth in SEQ ID NO: 47 or an amino acid sequence having at least 80% homology thereof; and the LCDR3 comprises an amino acid sequence as set forth in SEQ ID NO: 48 or an amino acid sequence having at least 80% homology thereof. In certain embodiments, the light chain variable region comprises an amino acid sequence as set forth in SEQ ID NO: 50 or an amino acid sequence having at least 80% homology thereof. In certain embodiments, the antibody or antigen-binding fragment thereof comprises a heavy chain variable region including HCDR1-HCDR3, wherein the HCDR1 comprises an amino acid sequence as set forth in SEQ ID NO: 43 or an amino acid sequence having at least 80% homology thereof; the HCDR2 comprises an amino acid sequence as set forth in SEQ ID NO: 44 or an amino acid sequence having at least 80% homology thereof, and the HCDR3 comprises an amino acid sequence as set forth in SEQ ID NO: 45 or an amino acid sequence having at least 80% homology thereof. In certain embodiments, the heavy chain variable region comprises an amino acid sequence as set forth in SEQ ID NO: 49 or an amino acid sequence having at least 80% homology thereof.

In certain embodiments, the targeting moiety is an antibody or antigen-binding fragment thereof that specially binds to and/or recognizes BCMA, and the antibody or antigen-binding fragment thereof comprises a light chain variable region including LCDR1-LCDR3, wherein the LCDR1 comprises an amino acid sequence as set forth in SEQ ID NO: 54 or an amino acid sequence having at least 80% homology thereof, the LCDR2 comprises an amino acid sequence as set forth in SEQ ID NO: 55 or an amino acid sequence having at least 80% homology thereof; and the LCDR3 comprises an amino acid sequence as set forth in SEQ ID NO: 56 or an amino acid sequence having at least 80% homology thereof. In certain embodiments, the light chain variable region comprises an amino acid sequence as set forth in SEQ ID NO: 58 or an amino acid sequence having at least 80% homology thereof. In certain embodiments, the antibody or antigen-binding fragment thereof comprises a heavy chain variable region including HCDR1-HCDR3 , wherein the HCDR1 comprises an amino acid sequence as set forth in SEQ ID NO: 51 or an amino acid sequence having at least 80% homology thereof; the HCDR2 comprises an amino acid sequence as set forth in SEQ ID NO: 52 or an amino acid sequence having at least 80% homology thereof; and the HCDR3 comprises an amino acid sequence as set forth in SEQ ID NO: 53 or an amino acid sequence having at least 80% homology thereof. In certain embodiments, the heavy chain variable region comprises an amino acid sequence as set forth in SEQ ID NO: 57 or an amino acid sequence having at least 80% homology thereof.

In certain embodiments, the targeting moiety is an antibody or antigen-binding fragment thereof that specially binds to and/or recognizes HER2, and the antibody or antigen-binding fragment thereof comprises a light chain variable region including LCDR1-LCDR3, wherein the LCDR1 comprises an amino acid sequence as set forth in SEQ ID NO: 70 or an amino acid sequence having at least 80% homology thereof; the LCDR2 comprises an amino acid sequence as set forth in SEQ ID NO: 71 or an amino acid sequence having at least 80% homology thereof; and the LCDR3 comprises an amino acid sequence as set forth in SEQ ID NO. 72 or an amino acid sequence having at least 80% homology thereof. In certain embodiments, the light chain variable region comprises an amino acid sequence as set forth in SEQ ID NO: 74 or an amino acid sequence having at least 80% homology thereof. In certain embodiments, the antibody or antigen-binding fragment thereof comprises a heavy chain variable region including HCDR1-HCDR3, wherein the HCDR1 comprises an amino acid sequence as set forth in SEQ ID NO: 67 or an amino acid sequence having at least 80% homology thereof, the HCDR2 comprises an amino acid sequence as set forth in SEQ ID NO: 68 or an amino acid sequence having at least 80% homology thereof; and the HCDR3 comprises an amino acid sequence as set forth in SEQ ID NO: 69 or an amino acid sequence having at least 80% homology thereof. In certain embodiments, the heavy chain variable region comprises an amino acid sequence as set forth in SEQ ID NO: 73 or an amino acid sequence having at least 80% homology thereof.

In certain embodiments, the targeting moiety is an antibody or a fragment for antigen-binding thereof that specially binds to and/or recognizes Mesothelin, and the antibody or antigen-binding fragment thereof comprises a light chain variable region including LCDR1-LCDR3, wherein the LCDR1 comprises an amino acid sequence as set forth in SEQ ID NO: 62 or an amino acid sequence having at least 80% homology thereof; the LCDR2 comprises an amino acid sequence as set forth in SEQ ID NO: 62 or an amino acid sequence having at least 80% homology thereof; and the LCDR3 comprises an amino acid sequence as set forth in SEQ ID NO: 64 or an amino acid sequence having at least 80% homology thereof. In certain embodiments, the light chain variable region comprises an amino acid sequence as set forth in SEQ ID NO: 66 or an amino acid sequence having at least 80% homology thereof. In certain embodiments, the antibody or antigen-binding fragment thereof comprises a heavy chain variable region including HCDR1-HCDR3, wherein the HCDR I comprises an amino acid sequence as set forth in SEQ ID NO: 59 or an amino acid sequence having at least 80% homology thereof; the HCDR2 comprises an amino acid sequence as set forth in SEQ ID NO: 60 or an amino acid sequence having at least 80% homology thereof, and the HCDR3 comprises an amino acid sequence as set forth in SEQ ID NO: 61 or an amino acid sequence having at least 80% homology thereof. In certain embodiments, the heavy chain variable region comprises an amino acid sequence as set forth in SEQ ID NO: 65 or an amino acid sequence having at least 80% homology thereof.

In certain embodiments, the targeting moiety is an antibody or antigen-binding fragment thereof that specially binds to and/or recognizes GPC3, and the antibody or antigen-binding fragment thereof comprises a light chain variable region including LCDR1-LCDR3, wherein the LCDR1 comprises an amino acid sequence as set forth in SEQ ID NO:38 or an amino acid sequence having at least 80% homology thereof, the LCDR2 comprises an amino acid sequence as set forth in SEQ ID NO: 39 or an amino acid sequence having at least 80% homology thereof; and the LCDR3 comprises an amino acid sequence as set forth in SEQ ID NO: 40 or an amino acid sequence having at least 80% homology thereof. In certain embodiments, the light chain variable region comprises an amino acid sequence as set forth in SEQ ID NO: 42 or an amino acid sequence having at least 80% homology thereof. In certain embodiments, the antibody or antigen-binding fragment thereof comprises a heavy chain variable region including HCDR1-HCDR3, wherein the HCDR1 comprises an amino acid sequence as set forth in SEQ ID NO: 35 or an amino acid sequence having at least 80% homology thereof; the HCDR2 comprises an amino acid sequence as set forth in SEQ ID NO: 36 or an amino acid sequence having at least 80% homology thereof; and the HCDR3 comprises an amino acid sequence as set forth in SEQ ID NO: 37 or an amino acid sequence having at least 80% homology thereof. In certain embodiments, the heavy chain variable region comprises an amino acid sequence as set forth in SEQ ID NO: 41 or an amino acid sequence having at least 80% homology thereof.

In certain embodiments, the targeting moiety comprises an amino acid sequence as set forth in any one of SEQ ID NO: 2, 4, 6, 8, and 10 or an amino acid sequence having at least 80% homology thereof. In certain embodiments, a nucleic acid molecule encoding the targeting moiety comprises a nucleic acid sequence as set forth in any one of SEQ ID NO: 1, 3, 5, 7, and 9 or an amino acid sequence having at least 80% homology thereof.

In certain embodiments, the method further comprises the step of isolating and obtaining peripheral blood mononuclear cells PBMC, CD3⁺T lymphocyte, CD8⁺T lymphocyte, CD4⁺T lymphocyte or regulatory T cells.

In certain embodiments, the method further comprises adding one or more T cell stimulatory factors into the isolated PBMC. In certain embodiments, the T cell stimulatory factors are selected from the group consisting of B lymphocyte surface antigens antibody, TNF antibody, intracellular polyester, and antibiotics. In certain embodiments, the T cell stimulatory factors are selected from the group consisting of CD3 antibody, CD28 antibody, 4-1BB antibody, CD80 antibody, CD86 antibody, PHA, PMA, and ionomycin.

In certain embodiments, the T cell stimulatory factors comprise CD3 antibody at a concentration of 1-10000 ng/mL. In certain embodiments, the T cell stimulatory factor comprise CD28 antibody at a concentration of 1-10000 ng/mL.

In certain embodiments, the method further comprises adding one or more cytokines into the isolated PBMC.

In certain embodiments, the cytokines comprise interleukins.

In certain embodiments, the interleukins comprise one or more selected from the group consisting of IL2, IL21, IL7, and IL15. In certain embodiments, the interleukins comprise IL2 at a concentration of 0.1-10000 U/mL. In certain embodiments, the interleukins comprise IL21 at a concentration of 0.01-1000 ng/mL. In certain embodiments, the interleukins comprise IL7 at a concentration of 0.01-1000 ng/mL. In certain embodiments, the interleukins comprise IL15 at a concentration of 0.01-1000 ng/mL.

In certain embodiments, the low density lipoprotein receptor-associated protein comprises one or more selected from the group consisting of the low density lipoprotein receptor-associated protein 1-12, and a functional fragment thereof. In certain embodiments, the low density lipoprotein receptor-associated protein or fragment thereof is derived from human.

In certain embodiments, the functional fragment comprises a fragment or Truncated protein of the low density lipoprotein receptor-associated protein with an activity of the low density lipoprotein receptor-associated protein. In certain embodiments, the low density lipoprotein receptor-associated protein comprises the low density lipoprotein receptor-associated protein 6 and its truncated protein, and/or the low density lipoprotein receptor-associated protein 5 and its truncated protein. In certain embodiments, the Truncated protein of the low density lipoprotein receptor-associated protein 6 comprises an intracellular region of the low density lipoprotein receptor-associated protein 6; and/or the truncated protein of the low density lipoprotein receptor-associated protein 5 comprises an intracellular region of the low density lipoprotein receptor-associated protein 5. In certain embodiments, the truncated protein of the low density lipoprotein receptor-associated protein 6 comprises a transmembrane region of the low density lipoprotein receptor-associated protein 6 and an LDLR region of the low density lipoprotein receptor-associated protein 6; and/or the truncated protein of the low density lipoprotein receptor-associated protein 5 comprises a transmembrane region of the low density lipoprotein receptor-associated protein S and an LDLR region of the low density lipoprotein receptor-associated protein 5. In certain embodiments, the low density lipoprotein receptor-associated protein or fragment thereof at least comprises the intracellular domain of the low density lipoprotein receptor-associated protein. For example, the intracellular domain of the low density lipoprotein receptor-associated protein 6 comprises the amino acid sequence from site 24 to site 243 in SEQ NO.24 or the amino acid sequence having at least 80% homology thereof. For example, the intracellular domain of the low density lipoprotein receptor-associated protein 5comprises the amino acid sequence from site 24 to site 231 in SEQ NO.28 or the amino acid sequence having at least 80% homology thereof. In certain embodiments, the low density lipoprotein receptor-associated protein or fragment thereof comprises an amino acid sequence as set forth in any one of SEQ ID NO: 22, 24, 26, and 28 or an amino acid sequence having at least 80% homology thereof. In certain embodiments, a nucleic acid molecule encoding the low density lipoprotein receptor-associated protein or fragment thereof comprises a nucleic acid sequence as set forth in any one of SEQ ID NO: 21, 23, 25, and 27 or an amino acid sequence having at least 80% homology thereof.

In another aspect, the present application provides a genetically modified immune cell, wherein the genetic modification op-regulates an expression of the low density lipoprotein receptor-associated protein or fragment thereof in the immune cells.

In certain embodiments, the genetically modified immune cell comprises lymphocyte. In certain embodiments, the genetically modified immune cell comprises genetically modified T cells. In certain embodiments, the genetically modified immune cell comprises genetically modified memory stem cell-like T cells (TSCM) and/or genetically modified central memory T cells (TCM). In certain embodiments, the TSCM is CCR7⁺ and/or CD62L⁺. In certain embodiments, the TSCM further has one or more properties selected from the group consisting of CD45RA⁺ or CD45RA⁻; CD45RO⁺ or CD45RO⁻, CD27⁺, CD28⁺, CD127⁺, CD122⁺, CD3⁺, CD4⁺, and CD8⁺.

In certain embodiments, the immune cell comprises genetically modified immune cells, and the genetically modified immune cells express a chimeric antigen receptor (CAR) or a T cell receptor (TCR).

In certain embodiments, the CAR comprises an intracellular domain, the intracellular domain comprises a signal transduction domain and/or a costimulatory domain. In certain embodiments, the signal transduction domain comprises a moiety selected from the group consisting of a signal transduction domain of CD3ζ, a signal transduction domain of CD3δ, and a signal transduction domain of CD3ε. In certain embodiments, the signal transduction domain comprises an amino acid sequence as set forth in SEQ ID NO: 18. In certain embodiments, a nucleic acid molecule encoding the signal transduction domain comprises a nucleic acid sequence as set forth in SEQ ID NO:17 or a nucleic acid sequence having at least 80% homology thereof.

In certain embodiments, the costimulatory domain comprises a moiety selected from the group consisting of a costimulatory domain of CD27, a costimulatory domain of CD28. and a costimulatory domain of 4-1BB. In certain embodiments, the costimulatory domain comprises an amino acid sequence as set forth in any one of SEQ ID NO:14, and SEQ ID NO:16 or an amino acid sequence having at least 80% homology thereof. In certain embodiments, a nucleic acid molecule encoding the costimulatory domain comprises a nucleic acid sequence as set forth in any one of SEQ ID NO:13, and SEQ ID NO:15 or a nucleic acid sequence having at least 80% homology thereof.

In certain embodiments, the CAR comprises a hinge region. In certain embodiments, the hinge region comprises a moiety selected from the group consisting of a hinge region of IgG4, a hinge region of IgG1, and a hinge region of CD8. In certain embodiments, the hinge region comprises an amino acid sequence as set forth in SEQ ID NO: 31 or an amino acid sequence having at least 80% homology thereof, In certain embodiments, a nucleic acid molecule encoding the hinge region comprises a nucleic acid sequence as set forth in SEQ ID NO: 32 or an amino acid sequence having at least 80% homology thereof.

In certain embodiments, the CAR comprises a transmembrane region. In certain embodiments, the transmembrane region comprises a moiety selected from the group consisting of a transmembrane region of CD8, a transmembrane region of CD28, and a transmembrane region of CD24. In certain embodiments, the transmembrane region comprises an amino acid sequence as set forth in SEQ ID NO: 33 or an amino acid sequence having at least 80% homology thereof. In certain embodiments, a nucleic acid molecule encoding the transmembrane region comprises a nucleic acid sequence as set forth in SEQ ID NO: 34 or a nucleic acid sequence having at least 80% homology thereof.

In certain embodiments, the CAR comprises a targeting moiety. In certain embodiments, the targeting moiety comprises ScFv. In certain embodiments, the targeting moiety specially binds to and/or recognizes tumor antigen. In certain embodiments, the targeting moiety specially binds to and/or recognizes a target selected from the group consisting of B lymphocyte surface antigens, TNF family members, HER family members, and GPC family members. In certain embodiments, the targeting moiety specially binds to and/or recognizes a target selected from the group consisting of CD19, BCMA, HER2, Mesothelin, and GPC3.

In certain embodiments, the targeting moiety is an antibody or antigen-binding fragment thereof that specially binds to and/or recognizes CD19, and the antibody or antigen-binding fragment thereof comprises a light chain variable region including LCDR1-LCDR3, wherein the LCDR1 comprises an amino acid sequence as set forth in SEQ ID NO: 46 or an amino acid sequence having at least 80% homology thereof; the LCDR2 comprises an amino acid sequence as set forth in SEQ ID NO: 47 or an amino acid sequence having at least 80% homology thereof; and the LCDR3 comprises an amino acid sequence as set forth in SEQ ID NO: 48 or an amino acid sequence having at least 80% homology thereof. In certain embodiments, the light chain variable region comprises an amino acid sequence as set forth in SEQ ID NO: 50 or an amino acid sequence having at least 80% homology thereof. In certain embodiments, the antibody or antigen-binding fragment thereof comprises a heavy chain variable region including HCDR1-HCDR3, wherein the HCDR1 comprises an amino acid sequence as set forth in SEQ ID NO: 43 or an amino acid sequence having at least 80% homology thereof the HCDR2 comprises an amino acid sequence as set forth in SEQ ID NO: 44 or an amino acid sequence having at least 80% homology thereof; and the HCDR3 comprises an amino acid sequence as set forth in SEQ ID NO: 45 or an amino acid sequence having at least 80% homology thereof. In certain embodiments, the heavy chain variable region comprises an amino acid sequence as set forth in SEQ ID NO: 49 or an amino acid sequence having at least 80% homology thereof.

In certain embodiments, the targeting moiety is an antibody or antigen-binding fragment thereof that specially binds to and/or recognizes BCMA, and the antibody or antigen-binding fragment thereof comprises a light chain variable region including LCDR1-LCDR3, wherein the LCDR1 comprises an amino acid sequence as set forth in SEQ ID NO: 54 or an amino acid sequence having at least 80% homology thereof; the LCDR2 comprises an amino acid sequence as set forth in SEQ ID NO: 55 or an amino acid sequence having at least 80% homology thereof. and the LCDR3 comprises an amino acid sequence as set forth in SEQ ID NO: 56 or an amino acid sequence having at least 80% homology thereof. In certain embodiments, the light chain variable region comprises an amino acid sequence as set forth in SEQ ID NO: 58 or an amino acid sequence having at least 80% homology thereof. In certain embodiments, the antibody or antigen-binding fragment thereof comprises a heavy chain variable region including HCDR1-HCDR3, wherein the HCDR1 comprises an amino acid sequence as set forth in SEQ ID NO: 51 or an amino acid sequence having at least 80% homology thereof; the HCDR2 comprises an amino acid sequence as set forth in SEQ ID NO: 52 or an amino acid sequence having at least 80% homology thereof, and the HCDR3 comprises an amino acid sequence as set forth in SEQ ID NO: 53 or an amino acid sequence having at least 80% homology thereof. In certain embodiments, the heavy chain variable region comprises an amino acid sequence as set forth in SEQ ID NO: 57 or an amino acid sequence having at least 80% homology thereof.

In certain embodiments, the targeting moiety is an antibody or antigen-binding fragment thereof that specially binds to and/or recognizes HER2, and the antibody or antigen-binding fragment thereof comprises a light chain variable region including LCDR1-LCDR3, wherein the LCDR1 comprises an amino acid sequence as set forth in SEQ ID NO: 70 or an amino acid sequence having at least 80% homology thereof; the LCDR2 comprises an amino acid sequence as set forth in SEQ ID NO: 71 or an amino acid sequence having at least 80% homology thereof; and the LCDR3 comprises an amino acid sequence as set forth in SEQ ID NO: 72 or an amino acid sequence having at least 80% homology thereof. In certain embodiments, the light chain variable region comprises an amino acid sequence as set forth in SEQ ID NO: 74 or an amino acid sequence having at least 80% homology thereof. In certain embodiments, the antibody or antigen-binding fragment thereof comprises a heavy chain variable region including HCDR1-HCDR3, wherein the HCDR1 comprises an amino acid sequence as set forth in SEQ ID NO: 67 or an amino acid sequence having at least 80% homology thereof the HCDR2 comprises an amino acid sequence as set forth in SEQ ID NO: 68 or an amino acid sequence having at least 80% homology thereof; and the HCDR3 comprises an amino acid sequence as set forth in SEQ ID NO: 69 or an amino acid sequence having at least 80% homology thereof. In certain embodiments, the heavy chain variable region comprises an amino acid sequence as set forth in SEQ ID NO: 73 or an amino acid sequence having at least 80% homology thereof.

In certain embodiments, the targeting moiety is an antibody or a fragment for antigen-binding thereof that specially binds to and/or recognizes Mesothelin, and the antibody or antigen-binding fragment thereof comprises a light chain variable region including LCDR1-LCDR3, wherein the LCDR1 comprises an amino acid sequence as set forth in SEQ ID NO: 62 or an amino acid sequence having at least 80% homology thereof; the LCDR2 comprises an amino acid sequence as set forth in SEQ ID NO: 63 or an amino acid sequence having at least 80% homology thereof; and the LCDR3 comprises an amino acid sequence as set forth in SEQ ID NO: 64 or an amino acid sequence having at least 80% homology thereof. In certain embodiments, the light chain variable region comprises an amino acid sequence as set forth in SEQ ID NO: 66 or an amino acid sequence having at least 80% homology thereof. In certain embodiments, the antibody or antigen-binding fragment thereof comprises a heavy chain variable region including HCDR1-HCDR3, wherein the HCDR1 comprises an amino acid sequence as set forth in SEQ ID NO: 59 or an amino acid sequence having at least 80% homology thereof; the HCDR2 comprises an amino acid sequence as set forth in SEQ ID NO: 60 or an amino acid sequence having at least 80% homology thereof; and the HCDR3 comprises an amino acid sequence as set forth in SEQ ID NO: 61 or an amino acid sequence having at least 80% homology thereof. In certain embodiments, the heavy chain variable region comprises an amino acid sequence as set forth in SEQ ID NO: 65 or an amino acid sequence having at least 80% homology thereof.

In certain embodiments, the targeting moiety is an antibody or antigen-binding fragment thereof that specially binds to and/or recognizes GPC3, and the antibody or antigen-binding fragment thereof comprises a light chain variable region including LCDR1-LCDR3, wherein the LCDR1 comprises an amino acid sequence as set forth in SEQ ID NO: 38 or an amino acid sequence having at least 80% homology thereof; the LCDR2 comprises an amino acid sequence as set forth in SEQ ID NO: 39 or an amino acid sequence having at least 80% homology thereof; and the LCDR3 comprises an amino acid sequence as set forth in SEQ ID NO:40 or an amino acid sequence having at least 80% homology thereof. In certain embodiments, the light chain variable region comprises an amino acid sequence as set forth in SEQ ID NO: 42 or an amino acid sequence having at least 80% homology thereof. In certain embodiments, the antibody or antigen-binding fragment thereof comprises a heavy chain variable region including HCDR1-HCDR3, wherein the HCDR1 comprises an amino acid sequence as set forth in SEQ ID NO: 35 or an amino acid sequence having at least 80% homology thereof; the HCDR2 comprises an amino acid sequence as set forth in SEQ ID NO: 36 or an amino acid sequence having at least 80% homology thereof; and the HCDR3 comprises an amino acid sequence as set forth in SEQ ID NO: 37 or an amino acid sequence having at least 80% homology thereof. In certain embodiments, the heavy chain variable region comprises an amino acid sequence as set forth in SEQ ID NO: 41 or an amino acid sequence having at least 80% homology thereof.

In certain embodiments, the targeting moiety comprises an amino acid sequence as set forth in any one of SEQ ID NO: 2, 4, 6, 8, and 10 or an amino acid sequence having at least 80% homology thereof. In certain embodiments, a nucleic acid molecule encoding the targeting moiety comprises a nucleic acid sequence as set forth in any one of SEQ ID NO: 1, 3, 5, 7, and 9) or a nucleic acid sequence having at least 80% homology thereof.

In certain embodiments, the low density lipoprotein receptor-associated protein comprises one or more selected from the group consisting of the low density lipoprotein receptor-associated protein 1-12, and a functional fragment thereof. In certain embodiments, the low density lipoprotein receptor-associated protein or fragment thereof is derived from human or mouse.

In certain embodiments, the functional fragment comprises a fragment or truncated protein of the low density lipoprotein receptor-associated protein with an activity of the low density lipoprotein receptor-associated protein. In certain embodiments, the low density lipoprotein receptor-associated protein comprises the low density lipoprotein receptor-associated protein 6 and truncated protein thereof, and/or the low density lipoprotein receptor-associated protein 5 and truncated protein thereof. In certain embodiments, the Truncated protein of the low density lipoprotein receptor-associated protein 6 comprises an intracellular region of the low density lipoprotein receptor-associated protein 6; and/or the Truncated protein of the low density lipoprotein receptor-associated protein 5 comprises an intracellular region of the low density lipoprotein receptor-associated protein 5. In certain embodiments, the truncated protein of the low density lipoprotein receptor-associated protein 6 comprises a transmembrane region of the low density lipoprotein receptor-associated protein 6 and an LDLR region of the low density lipoprotein receptor-associated protein 6; and/or the truncated protein of the low density lipoprotein receptor-associated protein 5 comprises a transmembrane region of the low density lipoprotein receptor-associated protein 5 and an LDLR region of the low density lipoprotein receptor-associated protein 5. In certain embodiments, the low density lipoprotein receptor-associated protein or fragment thereof comprises an amino acid sequence as set forth in any one of SEQ ID NO: 22, 24, 26, and 28 or an amino acid sequence having at least 80% homology thereof. In certain embodiments, a nucleic acid molecule encoding the low density lipoprotein receptor-associated protein or fragment thereof comprises a nucleic acid sequence as set forth in any one of SEQ ID NO: 21, 23, 25, and 27 or a nucleic acid sequence having at least 80% homology thereof.

In another aspect, the present application provides a composition including the genetically modified immune cell.

In certain embodiments, the composition further optionally comprises a pharmaceutically acceptable carrier.

In another aspect, the present application provides use of the genetically modified cell and/or the composition in manufacture of a drug for treating and/or preventing tumors.

In certain embodiments, the tumor is selected from the group consisting of liver cancer. lung cancer, leukemia, and mesothelioma.

In another aspect, the present application provides a method for preparing the composition including a following step: up-regulating an expression of the low density lipoprotein receptor-associated protein or fragment thereof in the genetically modified immune cell.

In certain embodiments, the method comprises a following step: introducing a vector up-regulating an expression of the low density lipoprotein receptor-associated protein or fragment thereof into the genetically modified immune cell. In certain embodiments, the vector is selected from the group consisting of retroviral vector, lentiviral vector, and transposon plasmid.

In certain embodiments, the low density lipoprotein receptor-associated protein or fragment thereof comprises an amino acid sequence as set forth in any one of SEQ ID NO: 22, 24, 26, and 28 or an amino acid sequence having at least 80% homology thereof.

In certain embodiments, the genetically modified immune cell comprises lymphocyte. In certain embodiments, the genetically modified immune cell expresses a chimeric antigen receptor (CAR). In certain embodiments, the method comprises a following step: isolating and activating the genetically modified immune cell, wherein the activating comprises an application of a T cell medium to the isolated genetically modified immune cell.

In certain embodiments, the T cell medium is one or more selected from the group consisting of DMEM medium, 1640 medium, MEM medium, and X-VIVO medium.

In certain embodiments, the method further comprises the application of T cell stimulatory factors to the genetically modified immune cell.

Persons skilled in the art can readily recognize other aspects and advantages of the present disclosure from the detailed description as below. The detailed description below only shows and describes exemplary embodiments of the present application. As persons skilled in the art will appreciate, the content of the present disclosure enables persons skilled in the art to modify the disclosed embodiments without departing from the spirit and scope of the invention involved in the present application. Correspondingly, the accompanying drawings and the description in the specification of the present application are only exemplary, rather than limited.

### BRIEF DESCRIPTION OF THE DRAWINGS

The particular features of the invention involved in the present application are as shown in the accompanying claims. With reference to the exemplary embodiments as detailedly described below and the accompanying drawings, the features and advantages of the invention involved in the present application can be better understood. The accompanying drawings are briefly described as follows:

FIG. 1 shows a schematic structural view of the CAR in the lentivirus according to the present application.

FIG. 2A, B, C and D show shows a result of the genetically modified immune cell according to the present application for promoting the production of memory immune cells.

FIG. 3 shows a result of the genetically modified immune cell according to the present application for inhibiting the differentiation of immune cells.

FIG. 4 shows a result of the low density lipoprotein receptor-associated protein or fragment thereof in the immune cells of the present application for promoting the proliferation of the immune cells of the present application.

FIGS. 5A-5B show a result of the genetically modified immune cell according to the present application for enhancing the releasing of cytokines.

FIG. 6 shows an anti-tumor effect of the genetically modified immune cell according to the present application.

FIG. 7 shows an effect of the genetically modified immune cell according to the present application for preventing the recurrence of a tumor.

FIG. 8 is a schematic diagram showing the sequence structure of CAR-T targeting GPC3 and overexpressing L6, extracellularly or intracellularly truncated/mutated L6, as well as L6 and L5 from different species (derived from human or murine).

FIG. 9 shows that overexpression of L6 and different intracellularly C-terminally truncated L6 (D1, D2, D3) can promote the proportion of memory cells Tscm in cultured GPC3-CAR-T cells.

FIG. 10 shows that overexpression of L6 and different intracellularly C-terminally truncated L6 (D1, D2, D3) can promote the proliferation of GPC-3 CAR-T cells after stimulation by target cells huh-7.

FIG. 11 shows that overexpression of L6 and different intracellularly C-terminally truncated L6 (D1, D2, D3) can promote the IL2 secretion of GPC-3 CAR-T cells after stimulation by target cells huh-7.

FIG. 12 shows that overexpression of L6 and different intracellularly C-terminally mutated L6 (M1, M2, M3, M4) can promote the proportion of memory cells Tscm in cultured GPC3-CAR-T cells.

FIG. 13 shows that overexpression of L6 and different intracellularly C-terminally mutated L6 (M1, M2, M3, M4) can promote the proliferation of GPC-3 CAR˜T cells after stimulation by target cells huh-7.

FIG. 14 shows that overexpression of LG and different intracellularly C-terminally mutated L6 (M1, M2, M3, M4) can promote the IL2 secretion of GPC-3 CAR-T cells after stimulation by target cells huh-7.

FIG. 15 shows that overexpression of L6 and intracellularly and extracellularly truncated (close to the transmembrane region) L6 (DI and DE) can promote the proportion of memory cells Tscm in cultured GPC3-CAR-T cells.

FIG. 16 shows that overexpression of L6 and intracellularly and extracellularly truncated (close to the transmembrane region) L6 (DI and DE) can promote the proliferation of GPC-3 CAR-T cells after stimulation by target cells huh-7.

FIG. 17 shows that overexpression of L6 and intracellularly and extracellularly truncated (close to the transmembrane region) L6 (DI and DE) can promote the IL2 secretion of GPC-3 CAR-T cells after stimulation by target cells huh-7.

FIG. 18 shows that overexpression of L6 and intracellularly and extracellularly truncated (close to the transmembrane region) L6 (DI and DE) can promote the proliferation of GPC-3 CAR-T cells after stimulation by target cells huh-7.

FIG. 19 shows that overexpression of LG and LS from different sources (derived from human or murine) can promote the proliferation of GPC-3 CAR-T cells after stimulation by target cells huh-7.

FIG. 20 shows that overexpression of L6 and L5 from different specied (derived from human or murine) can promote the IL2 secretion of GPC-3 CAR-T cells after stimulation by target cells huh-7.

FIG. 21 shows that overexpression of L6, L6 with transmembrane region replacement (CD8TM, LRP1TM for LRP6TM), LRP6 intracellular truncations (all the segments being intracellularly deleted from the first PPPS/TP motif), LRP6 intracellular mutations (all 5 PPPS/TP motifs being mutated into PPPAP), and LRP6 intracellular segment free structure can promote the cell proliferation in the process of CAR-T cell culture.

FIGS. 22A and 22B show that overexpression of L6, L6 with transmembrane region replacement (CD8TM, LRP1TM for LRP6TM), LRP6 intracellular truncations (all the segments being intracellularly deleted from the first PPPS/TP motif). LRP6 intracellular mutations (all 5 PPPS/TP motifs being mutated into PPPAP), and LRP6 free intracellular segment can promote the formation of memory T cells.

### DETAILED DESCRIPTION OF THE EMBODIMENTS

Hereinafter the embodiments of the present application are described by means of particular examples, and persons skilled in the art can readily understand other advantages and effects of the inventions as claimed in the present application based on the disclosure of the specification.

In the present application, the term “immune cell” generally refers to a cell involved in or associated with an immune response. The immune cell can comprise lymphocytes and a variety of phagocytes. The immune cells can further comprise naturally-occurring and genetically modified immune cells. In the present application, the genetically modified immune cell can express a chimeric antigen receptor (CAR). Of those, the lymphocyte can comprise T lymphocytes and B lymphocytes. In the present application, the immune cells can comprise T cells.

In the present application, the term “memory immune cells” generally refers to cells with immune memory. The immune memory can mean that a cell can produce a rapid and strong immune response when encountering a specific antigen to which it had produced a specific recognition and response. In the present application, the memory immune cells can comprise memory T cells. The memory T cells can be divided into memory stem cell-like T cells (TSCM) and central memory T cells (TCM).

In the present application, the term “differentiated immune cells” generally refers to immune cells with a certain degree of differentiation. For example, the differentiated immune cells can be T cells with a certain degree of differentiation. In the present application, the differentiated immune cells can be obtained by culturing the immune cells to a certain degree of differentiation. For example, the differentiated immune cells can comprise regulatory T cells (Treg).

In the present application, the term “regulatory T cells (Treg)” generally refers to a population of lymphocytes that negatively regulate the body's immune response. The molecular marker of the regulatory T cells can be a transcription factor, Foxp3⁺ or CD127⁺. In the present application, the regulatory T cells can be divided into two types, i.e., naturally-occurring or induced. Of those, the naturally-occurring species are CD4⁺CD25⁺ cells, and the induced species are T_{R}1 cells and T_{H}3 cells

In the present application, the term “subject susceptible to a tumor” generally refers to a subject with an increased probability of developing a tumor as compared with an ordinary subject. For example, the subject susceptible to a tumor can be a subject that has been cured with tumor, but has the risk of metastasis and recurrence. The subject susceptible to a tumor can also be a subject that has been diagnosed with tumor risk factors. For example, the risk factors can comprise some gene mutations (including deletions, additions or substitutions) that have been proven to be associated with tumors. In the present application, the subject susceptible to a tumor can also be a subject of prolonged exposure to the carcinogenic environment. For example, the carcinogenic environment can comprise strong radiation, high concentration of carcinogen.

In the present application, the term “genetic modification” generally refers to a alteration or modification occurring at the level of genetic structure. For example, the genetic modification can be a modification at the level of gene, transcription and/or translation. Alternatively, the genetic modification can comprise a change of any genetic property in an organism (including its tissues, cells, DNAs, mRNAs or proteins and fragments thereof, etc.). The genetic modification can comprise enabling the organism to express a particular protein or fragments thereof. For example, the genetic modification can comprise enabling the organism to express a vector of a particular protein or fragment thereof.

In the present application, the term “T cells” are also known as T lymphocytes, which are a subtype of leukocytes and plays a central role in cell-mediated immune. T cells can be distinguished from other lymphocytes (e.g., B cells and natural killer cells) by the T cell receptor present in the surface of cells. In the present application, the T cells can comprise memory stem cell-like T cells (TSCM) and central memory T cells (TCM).

In the present application, the term “memory stem cell-like T cells (TSCM)” generally refers to those that are in the early stage of differentiation of memory T cells, have the characteristics of stem cells, and have relatively strong multi-directional differentiation potential. After responding to antigen stimulation, TSCM cells can differentiate into central memory T cells (TCM), effector memory T cells (TEM), and effector T cells (TEF).

In the present application, the term “central memory T cells (TCM)” generally refers to T cells with long-term memory generated by the antigen activation of Native T cells. The biomarkers of TCM can comprise CD62L⁺ and CD45RO⁺. The central memory T cells can pass through the lymphatic shield, return to the lymph nodes, and exist in a state of being activated by the antigen.

In the present application, the term “T cells receptor” is also generally known as “TCR”, which generally refers to the molecular structure of T cells for specially recognizing and binding to the antigen peptide-MHC molecule. The T cell receptor can exist on the surface of T cells in the form of a complex with CD3 molecules. The TCR can be a heterodimer fixed on the cell membrane, most of them are composed of highly variable a subunits and β subunits linked by disulfide bonds, and a few of them are composed of γ and δ peptide chains. The TCR can comprise a variable region and a constant region, wherein the constant region can be close to the cell membrane, linking the transmembrane region and the end of the cell, while the variable region is responsible for recognizing the polypeptide/MHC complex.

In the present application, the term “chimeric antigen receptor” is also generally known as “CAR”, and” generally refers to a fusion protein including an extracellular domain capable of binding to an antigen and at least one intracellular domain. In the present application, the CAR can comprise an intracellular domain, and the intracellular domain comprises a signal transduction domain and/or a costimulatory domain. In the present application, a set of polypeptides of the CAR can be located in the same polypeptide chain (for example, including a chimeric fusion protein), or they can be discontinuous with each other, e.g., they can be located in different polypeptide chains. In the present application, the signals involved in induction can be transduced into the cytoplasm of T cells via CD3 and ζ chains. In the present application, the intracellular domain can comprise a primary signal transduction binding domain (for example, the main signal domain of CD3-zeta (ζ). In one aspect, the cytoplasmic signal domain can further comprise one or more costimulatory domains derived from at least one costimulatory molecule. For example, the costimulatory domain can be 4-1BB (i.e., CD137), CD27, ICOS and/or CD28. In the present application, the CAR can comprise a chimeric fusion protein, e.g., an optional leader sequence on the amino terminal (N-ter). Of those, the leader sequence optionally cleaves the antigen binding domain (e.g., ScFv), and localizes the CAR on the cell membrane during cell processing.

In the present application, the term “signal transduction domain” generally refers to a domain located inside a cell and capable of transducing signals. In the present application, the intracellular signal transduction domain can transduce signals into the cell For example, the intracellular signal transduction domain is an intracellular signaling domain of the chimeric antigen receptor. In the present application, the signal transduction domain can comprise a moiety selected from the group consisting of CD34, CD38, and a signal transduction domain of CD3c.

In the present application, the term “costimulatory domain” generally refers to a domain in the CAR that passes through the cell membrane, and is linked to the intracellular signal transduction domain and serves to transmitting signals. In the present application, the costimulatory domain can comprise a moiety selected from the group consisting of CD27, CD28, and a costimulatory domain of 4-1BB.

In the present application, the term “hinge region” generally refers to a junction region between an antigen binding region and an immune cell Fc receptor (FcR) binding region. For example, the hinge region can be a region between the heavy chain CH1 and CH2 of an immunoglobulin. In the present application, the hinge region can be a region located between scFv and T cell membrane. The hinge region can be derived from IgG1 or IgG4, and can also be derived from IgD or CD8. In the present application, a hinge region can comprise a moiety selected from the group consisting of a hinge region of IgG4, a hinge region of IgG1, and a hinge region of CD8.

In the present application, the term “transmembrane region” generally refers to a transmembrane region linking the extracellular antigen binding domain and the intracellular signal domain, which is generally composed of dimeric membrane proteins, comprises primarily CD3ζ, CD4, CDS, CD28, and the like, and can anchor the CAR structure on a T cell membrane. Different designs of the transmembrane region can affect the expression of the introduced CAR gene. In the present application, the transmembrane region can comprise a moiety selected from the group consisting of a transmembrane region of CD8, a transmembrane region of CD28, and a transmembrane region of CD24.

In the present application, the term “single chain antibody” (ScFv)” generally refers to an antibody formed by linking the heavy chain variable region and the light chain variable region via a linker. In the present application, the linker can be a linker peptide.

In the present application, the term “tumor antigen” generally refers to an antigenic substance present in or generated by a tumor cell that can have an ability of triggering an immune response in a host. For example, the tumor antigen can be protein, polypeptide, peptide, or fragment thereof that constitutes a part of the tumor cell and can induce tumor-specific cytotoxic T lymphocytes. In some embodiments, the term “tumor antigen” can also refer to cancer cell-associated biomolecules (e.g., proteins, carbohydrates, glycoproteins, etc.) that are specifically or preferentially or differentially expressed on tumor cells so as to provide cancer preferential or specific targets. For example, as compared with any other cells in the organism, the preferential expression can be a conventional preferential expression, or a preferential expression in a specific area of the organism (e.g., in particular organs or tissues). In the present application, the tumor antigen can comprise B lymphocyte surface antigens, TNF family members, HER family members, and OPC family members.

In the present application, the term “B lymphocyte surface antigen” generally refers to an antigen generated by a B lymphocyte during different phases and located on the surface of the B lymphocyte. For example, the B lymphocyte surface antigen can comprise CD10, CD19, CD20, CD21, CD22, CD23, CD24, CD37, CD3δ, CD39, and CD40. In the present application, B lymphocyte surface antigens can comprise CD19.

In the present application, the term “CD19” generally refers to the differentiation group 19 protein, which is an antigen determinant that can be detected on leukemia precursor cells. The accession number of human CD19 in UniProt/Swiss-Prot is P15391, and the accession number of the nucleotide sequence encoding human CD19 in GenBank is NM_001178098. In the present application, CD19 can also comprise a mutant protein or a functional fragment thereof, e.g., the point mutant, fragments, insertions, deletions, and splice variants of full-length wild-type CD19.

In the present application, the term “TNF family members” generally refers to a member belonging to the TNF (Tumor Necrosis Factor) family. TNF family members can comprise CD40LG (TNFSF5), CD70 (TNFSF7), EDA, FASLG (TNFSF6), LTA (TNFSF1), LTB (TNFSF3), TNFSF4 (OX40L), TNFSF8 (CD153), TNFSF9 (4-1BB), TNFSF10 (TRAIL), TNFSF11 (RANKL), TNFSF12 (TWEAK), TNFSF13, TNFSF13B, TNFSF14, TNFSF15, TNFSF17 (BCMA), and TNFSF18. In the present application, the TNF family members can comprise BCMA and 4-1BB.

In the present application, the term “BCMA” generally refers to B Cell Maturation Antigen (BCMA, CD269). BCMA is a member of the tumor necrosis factor receptor (TNF) superfamily, and can bind to B cell activating factor (BAFF) and proliferation-inducing ligand (APRIL). BCMA is commonly found on the surface of plasma cells in patients with multiple myeloma. The accession number of human BCMA in GenBank is BAB60895.1.

The term “CD137 protein”, also known as 4-1BB or TNFRS9, generally refers to a transmembrane protein of the tumor necrosis factor receptor superfamily (TNFRS). It is an activation-induced costimulatory molecule and an important modulator of immune response.

Studies have shown that CD137 agonistic monoclonal antibodies increase the expression of costimulatory molecules in many models, and significantly enhance cytolytic T lymphocyte responses and exert anti-tumor effects (see Vinay, Dass S., and Byoung S. Kwon. “4-1BB (CD137), an inducible costimulatory receptor, as a specific target for cancer therapy.” BMB reports 47.3 (2014): 122). The accession number of human CD137 in GenBank is NP_001552.2.

In the present application, the term “HER family member” generally refers to a member belonging to the HER (human epidermal growth factor receptor) family. HER family members can comprise EGFR (ErbB-1), HER2/c-neu (ErbB-2), Her3 (ErbB-3), and Her4 (ErbB-4). In the present application, the HER family member can comprise HER2.

In the present application, the term “HER2” generally refers to a human HER2 protein, which belongs to a member of the HER family. For example, see Semba et al., PNAS (USA) 82:6497-6501 (1985) and Yamamoto et al., Nature 319:230-234 (1986). The GenBank accession number of human HER2 can comprise XP_024306409.1.

In the present application, the term “GPC family member” generally refers to glypican. There are six types of glypicans identified in mammals, which are referred to as GPC1 to GPC6, respectively. Glypicans are abnormally expressed in cancers, including human hepatocellular carcinoma, ovarian cancer, mesothelioma, pancreatic cancer, glioma and breast cancer. In the present application, the GPC family member can comprise GPC3.

In the present application, the term “GPC3” generally refers to a protein encoded by glypican 3 (NCBI database gene ID: 2719), which is an early marker of liver cancer. GPC3 is highly expressed in hepatocellular carcinoma, and can be detected in tissues of patients with early hepatocellular carcinoma. The GenBank accession number of human GPC3 can be AAB87062.1.

In the present application, the term “Mesothelin (or briefly MSLN)” generally refers to a tumor differentiation antigen, which is generally present on the mesothelial cells lining the pleura, peritoneum, and pericardium. It is highly expressed in cancers, including malignant mesothelioma, pancreatic cancer, ovarian cancer, and lung adenocarcinoma. The GenBank accession number of human Mesothelin can be AAH09272.1.

In the present application, the term “peripheral blood mononuclear cell (PBMC)” generally refers to cells with mononuclear nuclei in peripheral blood. The peripheral blood mononuclear cells can comprise lymphocytes and monocytes. In the present application, the peripheral blood mononuclear cells can be separated using Ficoll-hypaque (dextran-meglumine diatrizoate) density gradient centrifugation method according to the specific gravity difference of various components in the blood.

In the present application, the term “T cell activating factor” generally refers to a substance that promotes the activation and proliferation of T cells. In the present application, the T cell activating factor can comprise B lymphocyte surface antigen antibodies, TNF antibodies, intracellular polyesters and/or antibiotics. In the present application, the T cell stimulating factor can be selected from the following group: CD3 antibody, CD28 antibody, 4-1BB antibody, CD80 antibody, CD86 antibody, PHA, PMA, and ionomycin.

In the present application, the term “B lymphocyte surface antigen antibody” generally refers to an antibody that specifically binds to the surface antigen of B lymphocyte. In the present application, B lymphocyte surface antigen antibodies can comprise CD3 antibodies, CD28 antibodies, CD80 antibodies, and CD86 antibodies.

In the present application, the term “TNF antibody” generally refers to an antibody that specifically binds to a member of the TNF family. In the present application, the TNF antibody can comprise 4-1BB antibody.

In the present application, the term “intracellular polyester” generally refers to a naturally occurring polymeric biomaterial synthesized by microorganisms and present in the form of inclusion bodies in cells. Intracellular polyester has good biological properties. In the present application, the intracellular polyester can comprise polyhydroxyalkanoate (PHA).

In the present application, the term “antibiotic” generally refers to a metabolite produced by microorganisms, animals or plants that has anti-pathogenic or other activities and can interfere with the development and functionization of other living cells. In the present application, the antibiotics can comprise β-lactams, aminoglycosides, amido alcohols, macrolides, polypeptides, nitroimidazoles and tetracyclines. For example, the antibiotic can comprise ionomycin.

In the present application, the term “PHA” generally refers to polyhydroxy fatty acid esters, which belong to intracellular polyesters synthesized by various bacteria, and can exist in the form of discontinuous inclusion bodies in the cytoplasm in vivo. PHA can have physical and chemical properties similar to synthetic plastics, as well as biodegradability, biocompatibility, optical activity, piezoelectricity, and gas barrier properties.

In the present application, the term “PMA” generally refers to propylene glycol methyl ether acetate (Phorbol-12-myristate-13-acetate).

In the present application, the term “CD3 antibody” generally refers to an antibody or antigen-binding fragment thereof that specifically binds to CD3. CD3 can be an important differentiation antigen on the T cell membrane and can transmit the signal of T cell activation. The CD3 antibody can be huOKT3g1 or HuM291.

In the present application, the term “CD28 antibody” generally refers to an antibody or antigen-binding fragment thereof that specifically binds to CD28. Human CD28 is located at 2q33 and has similar exons and introns to CTLA4. The ligands of the two are the B7 family. including B7-1 (CD80) and B7-2 (CD86).

In the present application, the term “cytokine” generally refers to a class of small molecular proteins that are synthesized and secreted by immune cells (e.g., monocytes, macrophages, T cells, B cells, NK cells, etc.) and some non-immune cells (endothelial cells, epidermal cells, fibroblasts, etc.) under stimulus and have broadly biological activities. The cytokines can have various functions such as regulating innate immunity and adaptive immunity. hematogenesis, cell growth, APSC pluripotent cells, and repairing damaged tissues. In the present application, the cytokines can comprise interleukins, interferons, tumor necrosis factor superfamily, colony stimulating factors, chemokines, and growth factors. For example, the cytokine can be an interleukin.

In the present application, the term “interleukin” generally refers to secreted proteins or signal molecules that can promote the development and differentiation of T and/or B lymphocytes and/or hematopoietic cells. Interleukins can be synthesized by helper CD4T lymphocytes, as well as by monocytes, macrophages and endothelial cells. In the present application, the term “interleukin” can comprise full-length interleukins or fragments (for example, Truncated proteins) or variants thereof, which substantially retain the biological activity of the corresponding wild-type interleukin (e.g., having a biological activity of at least 80%, at least 90%, at least 95%, at least 98%, at least 99%, or even at least 100% of the biological activity of the corresponding wild-type interleukin). The interleukin as used herein can be from any mammalian species. In certain embodiments, the interleukin is from a species selected from humans, horses, cattle, mice, pigs, rabbits, cats, dogs, rats, goats, sheep, and non-human primates. In certain embodiments, the interleukin can be a mutant form. For example, in the present application, the cytokine comprises one or more selected from the group consisting of IL2, IL21, IL7 and IL15.

in the present application, the term “IL2” generally refers to the T cell growth factor, TCGF. It is produced by T cells and exerts benefits in an autocrine and paracrine manner. It can activate T cells and promote the production of cytokines, stimulate the proliferation of NK cells and induce the production of LAK cells, promote the proliferation of B cells and secrete antibodies, and can also activate macrophages.

In the present application, the term “IL21” is homologous in spatial structure with IL-2, IL-4, and IL-15, and can generally promote the proliferation and differentiation of bone marrow NK cells, and synergistically stimulate the proliferation of B cells with anti-CD40 antibodies, and synergistically stimulate the proliferation of T cells with anti-CD3 antibody.

In the present application, the term “IL7” generally refers to a glycoprotein secreted by bone marrow stromal cells, and its gene is located on chromosome 8. The target cells of IL7 are lymphocytes, especially capable of promoting the growth of B progenitor cells, thymocytes and peripheral mature T cells from human or mouse bone marrow. At higher concentrations, IL-7 can also enhance the cytotoxic activity of macrophages and induce monocytes to secrete cytokines.

In the present application, the term “IL15” generally refers to the production of a variety of cells, such as activated monocytes-macrophages, epidermal cells and fibroblasts, and has many similarities with IL2. IL15 is a member of the IL2 family. It regulates the activation and proliferation of T and NK cells by changing the expression of Bcl-1 family members (such as Bcl-2 and Bcl-XL), and can also induce B cell proliferation.

In the present application, the term “low density lipoprotein receptor-related protein” (LRP) generally refers to an intrinsic protein containing 839 amino acids (by removing 21 amino acid signal peptides). It is embedded in the outer phospholipid layer of LDL (Low density lipoprotein) particles, and belongs to an endocytic receptor that can mediate the endocytosis of cholesterol-rich LDL. It is a member of the low density lipoprotein receptor (LDLR) gene family. The LRP is most prominently expressed in bronchial epithelial cells and adrenal and cortical tissues. In the present application, the low-density lipoprotein receptor-related protein can comprise one or more selections from the group consisting of low-density lipoprotein receptor-related proteins 1-12 or a Truncated protein thereof.

In the present application, the terms “low density lipoprotein receptor-related protein 6” (LRP-6) and “low density protein receptor-related protein 5” (LRP-5) generally refer to unique subgroups of the low density lipoprotein receptor (LDLR) family. The accession number of human LRP-6 in UniProt is O75581. The accession number of human LRP-5 in UniProt is O75197.

In the present application, the term “truncated protein” generally refers to a truncated protein. The truncated protein can be obtained by proteolysis or by manipulating the structural gene to eliminate the N- or C-terminal of a protein. Alternatively, the truncated protein can be obtained by prematurely terminating translation due to the presence of a stop codon in the structural gene caused by a nonsense mutation.

In the present application, the term “intracellular region” generally refers to a domain of protein located inside the cell membrane. In the present application, the intracellular domain can refer to a domain of the low density lipoprotein receptor-associated protein in the cell membrane. In the present application, the intracellular region can comprise the sequence from site 24 to site 243 in SEQ NO.24 or an amino acid sequence having at least 80% homology thereof, or comprise the sequence from site 24 to site 231 in SEQ NO.28 or an amino acid sequence having at least 80% homology thereof.

In the present application, the term “transmembrane region” generally refers to a domain of protein located across the cell membrane. In the present application, the transmembrane region can refer to a domain of the low density lipoprotein receptor-associated protein located across the cell membrane. The transmembrane region can consist of 23 hydrophobic amino acids. This domain primarily provides an anchoring function for the binding of LDLR on the cell membrane. In the present application, the transmembrane region can comprise the sequence from site 1 to site 23 in SEQ NO. 24 or an amino acid sequence having at least 80% homology thereof, or the sequence from site 1 to site 23 in SEQ NO. 28 or an amino acid sequence having at least 80% homology thereof.

In the present application, the term “LDLR region” generally refers to a domain of the low-density lipoprotein receptor-related protein close to the N segment outside the transmembrane region. This domain can have a function of enhancing Wnt signal. In the present application, the LDLR region can comprise the sequence from site 5 to site 119 in SEQ NO. 22 or an amino acid sequence having at least 80% homology thereof, or the sequence from site 4 to site 119 in SEQ NO. 26 or an amino acid sequence having at least 80% homology thereof.

In the present application, the term “ribosome skip site” is also called internal ribosome entry site (IRES), and generally refers to a nucleotide sequence located in the middle of the mRNA sequence for translation initiation. The ribosome skip site can allow initiation of translation in a cap-independent manner. IRES is generally located in the 5′UTR. In the present application, the ribosome skip site can comprise the sequence from site 1 to site 578 in SEQ NO.29.

In the present application, the term “2A sequence” generally refers to a self-cleaving amino acid sequence that does not rely on protease. The 2A sequence can facilitate transcription to produce two proteins. In the present application, the 2A sequence can comprise the sequence from site 1 to site 54 in SEQ NO.75

In the present application, the ten “pharmaceutically acceptable carrier” can comprise buffers, antioxidants, preservatives, low molecular weight polypeptides, proteins, hydrophilic polymers, amino acids, sugars, chelating agents, counter ions, metal complexes and/or non-ionic surfactants, etc.

In the present application, the term “retroviral vector” generally refers to an RNA virus that can be subject to reverse transcription in infected cells to produce a complementary DNA strand, and use the single DNA strand as a template to synthesize a second DNA strand which is then incorporated into the cell genomic DNA. The retroviral vector can use enzymes in the host cell to transcribe and replicate RNA to synthesize protein by itself, and then package the viruses which are released from the cell and become infectious viruses. The retrovirus has high transduction efficiency and can effectively increase the gene transfection rate.

In the present application, the term “lentiviral vector” refers to a gene therapeutic vector developed on the basis of HIV-I (Human Immunodeficiency Type I Virus). The lentiviral vector can infect both dividing cells and non-dividing cells. It can effectively infect almost all mammalian cells including neuronal cells and hepatocytes with high infection efficiency. Lentivirus can effectively integrate foreign genes into the host chromosome to achieve persistent expression.

In the present application, the term “transposon plasmid” generally refers to a basic unit that exists on chromosomal DNA and can autonomously replicate and shift. The transposon plasmid can “jump” from one site to another in the genome through a series of processes such as cutting and reintegration.

In the present application, the term “tumor” generally refers to a neogrowth formed by a certain cell in a local tissue of an organism which loses the normal regulation for its growth at the level of gene under various carcinogenic factors, thereby resulting in its clonal abnormal proliferation. Such neogrowth is also called neoplasm because it is mostly presented as a space-occupying massive protrusion. In the present application, the tumor can comprise solid tumors and non-solid tumors. In the present application, the tumor can comprise liver cancer, lung cancer, leukemia and mesothelioma.

In the present application, the term “significant(ly) increase” generally refers to an increase in levels (e.g., protein expression, cell number), P<0.05, P<0.04, P<0.03, P<0.02,P<0.01, P<0,005 or P <0.001. Alternatively, the significant increase can refer to an increase of 5% or more, 10% or more, 20% or more, 30% or more, 40% or more, 50% or more, 60% or more, 70% or more, 80% or more, 90% or more, 100% or more.

In the present application, the term “substantial(ly) no increase” generally means that the level (e.g., protein expression, cell number) is the same as the original, or increased by 3% or less, increased by 2% or less, increased by 1% or less, increased by 0.5% or less, increased by 0.4% or less, increased by 0.3% or less, increased by 0.2% or less, increased by 0.1% or less.

In the present application, the term “about” generally refers to variations within a range of the specified value=0.5-10%, e.g., a variation within a range of the specified value=0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%

### Low Density Lipoprotein Receptor-Associated Protein or Fragment Thereof

In the present application, the low density lipoprotein receptor-associated protein can comprise one or more selections from the group consisting of the low density lipoprotein receptor-associated protein 1-12, and a functional fragment thereof.

In the present application, the low density lipoprotein receptor-associated protein or fragment thereof can be derived from mammals, for example, can be derived from human, macaque, rat, and mouse.

In the present application, the functional fragment can comprise a fragment or Truncated protein of the low density lipoprotein receptor-associated protein with an activity of the low density lipoprotein receptor-associated protein. For example, the low density lipoprotein receptor-associated protein can comprise the low density lipoprotein receptor-associated protein 6 and truncated protein thereof, and/or the low density lipoprotein receptor-associated protein 5 and truncated protein thereof.

In the present application, the truncated protein of the low density lipoprotein receptor-associated protein 6 can comprise an intracellular region of the low density lipoprotein receptor-associated protein 6; and/or the truncated protein of the low density lipoprotein receptor-associated protein 5 can comprise the intracellular region of the low density lipoprotein receptor-associated protein 5. Alternatively, the truncated protein of the low density lipoprotein receptor-associated protein 6 can comprise a transmembrane region of the low density lipoprotein receptor-associated protein 6 and an LDLR region of the low density lipoprotein receptor-associated protein 6; and/or the truncated protein of the low density lipoprotein receptor-associated protein 5 can comprise a transmembrane region of the low density lipoprotein receptor-associated protein 5 and an LDLR region of the low density lipoprotein receptor-associated protein 5.

In the present application, the low density lipoprotein receptor-associated protein (LRP) may comprise the intracellular region of LRP or its functional fragment.

For example, the C terminal of the intracellular region can be truncated. For example, the five PPPS/TP motifs at C terminals can be truncated or mutated. For example, at least one PPPS/TP motif at C terminals can be truncated or mutated.

For example, the LRP or the fragment thereof may comprise a transmembrane region. For example, the transmembrane region may be derived from the LRP1 transmembrane region, the LRP5 transmembrane region, the LRP6 transmembrane region, the CD8 transmembrane region.

For example, the LRP may comprise a free LRP. For example, the LRP may do not comprise a transmembrane region.

In the present application, the low density lipoprotein receptor-associated protein 6 or fragment thereof at least comprise the amino acid sequence from site 24 to site 243 in SEQ NO.24 or the amino acid sequence having at least 80% homology thereof.

In the present application, the low density lipoprotein receptor-associated protein 5 or fragment thereof at least comprise the amino acid sequence from site 24 to site 231 in SEQ NO.28 or the amino acid sequence having at least 80% homology thereof.

In the present application, the low density lipoprotein receptor-associated protein or fragment thereof comprises an amino acid sequence as set forth in any one of SEQ ID NO: 22, 24, 26, and 28 or an amino acid sequence having at least 80% homology thereof.

In the present application, the low density lipoprotein receptor-associated protein or fragment thereof comprises an amino acid sequence as set forth in any one of SEQ ID NO: 22, 24, 26, 28, 75-85, and 89-91 or an amino acid sequence having at least 80% homology thereof

In the present application, a nucleic acid molecule encoding the low density lipoprotein receptor-associated protein or fragment thereof comprises a nucleic acid sequence as set forth in any one of SEQ ID NO: 21, 23, 25, and 27 or a nucleic acid sequence having at least 80% homology thereof.

### Genetically Modified Immune Cells

The present application provides a genetically modified immune cell, wherein the expression of the low density lipoprotein receptor-associated protein or fragment thereof is upregulated in the immune cell.

In some embodiments, the proliferation of the genetically modified immune cell is promoted compared with unmodified immune cell, for example, the proliferation of the genetically modified immune cell is promoted at least about 10%, 15%, 20%, 25%, 30%, 35%, 40%, 45%, 50%, 55%, 60%, 65%, 70%, 75%, 80%, 85%, 90%, 95%, 100% or more compared with unmodified immune cell.

In some embodiments, the production of the memory cell from the genetically modified immune cell is promoted compared with unmodified immune cell, for example, the production of the memory cell from the genetically modified immune cell is promoted at least about 10%, 15%, 20%, 25%, 30%, 35%, 40%, 45%, 50%, 55%, 60%, 65%, 70%, 75%, 80%, 85%, 90%, 95%, 100% or more compared with unmodified immune cell.

In some embodiments, the differentiation of the genetically modified immune cell to a regulatory T cell (Treg) is inhibited compared with unmodified immune cell.

In some embodiments, the release of a cytokine from the genetically modified immune cell is increased compared with unmodified immune cell, for example, the release of the cytokine increased at least about 10%, 15%, 20%, 25%, 30%, 35%, 40%, 45%, 50%, 55%, 60%, 65%, 70%, 75%, 80%, 85%, 90%, 95%, 100% or more compared with unmodified immune cell.

In some embodiments, the ability of killing the tumor cell of the genetically modified immune cell is enhanced compared with unmodified immune cell, for example, the ability of killing tumor cells of the genetically modified immune cell is enhanced at least about 10%, 15%, 20%, 25%, 30%, 35%, 40%, 45%, 50%, 55%, 60%, 65%, 70%, 75%, 80%, 85%, 90%, 95%, 100% or more compared with unmodified immune cell.

In the present application, the genetically modified immune cell can comprise lymphocyte. In the present application, the genetically modified immune cell comprises genetically modified T cells. In the present application, the genetically modified immune cell comprises genetically modified memory stem cell-like T cells (TSCM) and/or genetically modified central memory T cells (TCM). In the present application, the TSCM comprises CCR7 and/or CD62L⁺. In the present application, the TSCM can further have one or more properties selected from the group consisting of CD45RA⁺ or CD45RA⁻, CD45RO⁺ or CD45RO⁻, CD27⁺, CD28⁺, CD127⁺, CD122⁺, CD3⁺, CD4⁺, and CD8⁺.

In the present application, the immune cells can comprise genetically modified immune cells, and the genetically modified immune cells express chimeric antigen receptor (CAR) or T cell receptor (TCR).

In the present application, the CAR can comprise an intracellular domain, and the intracellular domain can comprise a signal transduction domain and/or a costimulatory domain.

For example, the signal transduction domain can comprise a moiety selected from the group consisting of a signal transduction domain of CD3ζ, a signal transduction domain of CD3δ, and a signal transduction domain of CD3ε. For example, the signal transduction domain can comprise an amino acid sequence as set forth in SEQ ID NO: 18 or an amino acid sequence having at least 80% homology thereof, a nucleic acid molecule encoding the signal transduction domain can comprise a nucleic acid sequence as set forth in SEQ ID NO:17 or a nucleic acid sequence having at least 80% homology thereof.

For example, the costimulatory domain can comprise a moiety selected from the group consisting of a costimulatory domain of CD27, a costimulatory domain of CD28, and a costimulatory domain of 4-1BB. For example, the costimulatory domain can comprise an amino acid sequence as set forth in any one of SEQ ID NO:14, and SEQ ID NO: 16 or an amino acid sequence having at least 80% homology thereof, a nucleic acid molecule encoding the costimulatory domain can comprise a nucleic acid sequence as set forth in any one of SEQ ID NO: 13, and SEQ ID NO:15 or a nucleic acid sequence having at least 80% homology thereof.

In the present application, the CAR can comprise a hinge region. For example, the hinge region can comprise a moiety selected from the group consisting of a hinge region of IgG4, a hinge region of IgG1, and a hinge region of CDS. For example, the hinge region can comprise an amino acid sequence as set forth in SEQ ID NO: 31 or an amino acid sequence having at least 80% homology thereof, a nucleic acid molecule encoding the hinge region can comprise a nucleic acid sequence as set forth in SEQ ID NO: 32 or a nucleic acid sequence having at least 80% homology thereof.

In the present application, the CAR can comprise a transmembrane region. For example, the transmembrane region can comprise a moiety selected from the group consisting of a transmembrane region of CD8, a transmembrane region of CD28, and a transmembrane region of CD24. For example, the transmembrane region can comprise an amino acid sequence as set forth in SEQ ID NO: 33 or an amino acid sequence having at least 80% homology thereof, a nucleic acid molecule encoding the transmembrane region can comprise a nucleic acid sequence as set forth in SEQ ID NO: 34 or a nucleic acid sequence having at least 80% homology thereof.

In the present application, the CAR can comprise a targeting moiety. In the present application, the targeting moiety can comprise antibody or antigen binding fragment. The antigen binding fragment can be selected from the group consisting of Fab, Fab′, F(ab)2, F(ab′)2, Fv, and ScFv fragments. For example, the targeting moiety can be ScFv.

In the present application, the targeting moiety can specially bind to and/or recognize tumor antigen. For example, the targeting moiety can specially bind to and/or recognize a target selected from the group consisting of B lymphocyte surface antigens, TNF family members, HER family members, and GPC family members. Alternatively, the targeting moiety can specially bind to and/or recognize a target selected from the group consisting of CD19, BCMA, HER2, Mesothelin, and GPC3.

In the present application, the targeting moiety can be an antibody or antigen-binding fragment thereof that specially binds to and/or recognizes CD19. The antibody or antigen-binding fragment thereof can comprise a light chain variable region, the light chain variable region can comprise LCDR1-LCDR3, wherein the LCDR1 can comprise an amino acid sequence as set forth in SEQ ID NO: 46 or an amino acid sequence having at least 80% homology thereof: the LCDR2 can comprise an amino acid sequence as set forth in SEQ ID NO: 47 or an amino acid sequence having at least 80% homology thereof; and the LCDR3 can comprise an amino acid sequence as set forth in SEQ ID NO: 48 or an amino acid sequence having at least 80% homology thereof. The light chain variable region can comprise an amino acid sequence as set forth in SEQ ID NO: 50 or an amino acid sequence having at least 80% homology thereof. In the present application, the antibody or antigen-binding fragment thereof can comprise a heavy chain variable region, the heavy chain variable region can comprise HCDR1-HCDR3, wherein the HCDR1 can comprise an amino acid sequence as set forth in SEQ ID NO: 43 or an amino acid sequence having at least 80% homology thereof; the HCDR2 can comprise an amino acid sequence as set forth in SEQ ID NO: 44 or an amino acid sequence having at least 80% homology thereof; and the HCDR3 can comprise an amino acid sequence as set forth in SEQ ID NO: 45 or an amino acid sequence having at least 80% homology thereof. The heavy chain variable region can comprise an amino acid sequence as set forth in SEQ ID NO: 49 or an amino acid sequence having at least 80% homology thereof.

In the present application, the targeting moiety is an antibody or antigen-binding fragment thereof that specially binds to and/or recognizes BCMA, the antibody or antigen-binding fragment thereof can comprise a light chain variable region, the light chain variable region can comprise LCDR1-LCDR3, wherein the LCDR1 can comprise an amino acid sequence as set forth in SEQ ID NO: 54 or an amino acid sequence having at least 80% homology thereof; the LCDR2 can comprise an amino acid sequence as set forth in SEQ ID NO: 55 or an amino acid sequence having at least 80% homology thereof; and the LCDR3 can comprise an amino acid sequence as set forth in SEQ ID NO: 56 or an amino acid sequence having at least 80% homology thereof. The light chain variable region can comprise an amino acid sequence as set forth in SEQ ID NO: 58 or an amino acid sequence having at least 80% homology thereof. In the present application, the antibody or antigen-binding fragment thereof can comprise a heavy chain variable region, the heavy chain variable region can comprise HCDR1-HCDR3, wherein the HCDR1 can comprise an amino acid sequence as set forth in SEQ ID NO: 51 or an amino acid sequence having at least 80% homology thereof; the HCDR2 can comprise an amino acid sequence as set forth in SEQ ID NO: 52 or an amino acid sequence having at least 80% homology thereof; and the HCDR3 can comprise an amino acid sequence as set forth in SEQ ID NO: 53 or an amino acid sequence having at least 80% homology thereof. The heavy chain variable region can comprise an amino acid sequence as set forth in SEQ ID NO: 57 or an amino acid sequence having at least 80% homology thereof.

In the present application, the targeting moiety is an antibody or antigen-binding fragment thereof that specially binds to and/or recognizes HER2, the antibody or antigen-binding fragment thereof can comprise a light chain variable region, the light chain variable region can comprise LCDR1-LCDR3, wherein the LCDR1 can comprise an amino acid sequence as set forth in SEQ ID NO: 70 or an amino acid sequence having at least 80% homology thereof, the LCDR2 can comprise an amino acid sequence as set forth in SEQ ID NO: 71 or an amino acid sequence having at least 80% homology thereof; and the LCDR3 can comprise an amino acid sequence as set forth in SEQ ID NO: 72 or an amino acid sequence having at least 80% homology thereof. The light chain variable region can comprise an amino acid sequence as set forth in SEQ ID NO: 74 or an amino acid sequence having at least 80% homology thereof. In the present application, the antibody or antigen-binding fragment thereof can comprise a heavy chain variable region, the heavy chain variable region can comprise HCDR1-HCDR3, wherein the HCDR1 can comprise an amino acid sequence as set forth in SEQ ID NO: 67 or an amino acid sequence having at least 80% homology thereof; the HCDR2 can comprise an amino acid sequence as set forth in SEQ ID NO: 68 or an amino acid sequence having at least 80% homology thereof; and the HCDR3 can comprise an amino acid sequence as set forth in SEQ ID NO: 69 or an amino acid sequence having at least 80% homology thereof. The heavy chain variable region can comprise an amino acid sequence as set forth in SEQ ID NO: 73 or an amino acid sequence having at least 80% homology thereof.

In the present application, the targeting moiety is an antibody or a fragment for antigen-binding thereof that specially binds to and/or recognizes Mesothelin, the antibody or antigen-binding fragment thereof can comprise a light chain variable region, the light chain variable region can comprise LCDR1-LCDR3, wherein the LCDR1 can comprise an amino acid sequence as set forth in SEQ ID NO: 62 or an amino acid sequence having at least 80% homology thereof; the LCDR2 can comprise an amino acid sequence as set forth in SEQ ID NO: 63 or an amino acid sequence having at least 80% homology thereof; and the LCDR3 can comprise an amino acid sequence as set forth in SEQ ID NO:64 or an amino acid sequence having at least 80% homology thereof. The light chain variable region can comprise an amino acid sequence as set forth in SEQ ID NO: 66 or an amino acid sequence having at least 80% homology thereof. The antibody or antigen-binding fragment thereof can comprise a heavy chain variable region, the heavy chain variable region can comprise HCDR1-HCDR3, wherein the HCDR1 can comprise an amino acid sequence as set forth in SEQ ID NO: 59 or an amino acid sequence having at least 80% homology thereof; the HCDR2 can comprise an amino acid sequence as set forth in SEQ ID NO: 60 or an amino acid sequence having at least 80% homology thereof; and the HCDR3 can comprise an amino acid sequence as set forth in SEQ ID NO: 61 or an amino acid sequence having at least 80% homology thereof. The heavy chain variable region can comprise an amino acid sequence as set forth in SEQ ID NO: 65 or an amino acid sequence having at least 80% homology thereof.

In the present application, the targeting moiety is an antibody or antigen-binding fragment thereof that specially binds to and/or recognizes GPC3, the antibody or antigen-binding fragment thereof can comprise a light chain variable region, the light chain variable region can comprise LCDR1-LCDR3, wherein the LCDR1 can comprise an amino acid sequence as set forth in SEQ ID NO: 38 or an amino acid sequence having at least 80% homology thereof; the LCDR2 can comprise an amino acid sequence as set forth in SEQ ID NO:39 or an amino acid sequence having at least 80% homology thereof; and the LCDR3 can comprise an amino acid sequence as set forth in SEQ ID NO: 40 or an amino acid sequence having at least 80% homology thereof. The light chain variable region can comprise an amino acid sequence as set forth in SEQ ID NO: 42 or an amino acid sequence having at least 80% homology thereof. In the present application, the antibody or antigen-binding fragment thereof can comprise a heavy chain variable region, the heavy chain variable region can comprise HCDR1-HCDR3, wherein the HCDR1 can comprise an amino acid sequence as set forth in SEQ ID NO: 35 or an amino acid sequence having at least 80% homology thereof; the HCDR2 can comprise an amino acid sequence as set forth in SEQ ID NO: 36 or an amino acid sequence having at least 80% homology thereof; and the HCDR3 can comprise an amino acid sequence as set forth in SEQ ID NO: 37 or an amino acid sequence having at least 80% homology thereof. The heavy chain variable region can comprise an amino acid sequence as set forth in SEQ ID NO: 41 or an amino acid sequence having at least 80% homology thereof.

In the present application, the targeting moiety can comprise an amino acid sequence as set forth in any one of SEQ ID NO: 2, 4, 6, 8, and 10 or an amino acid sequence having at least 80% homology thereof. In the present application, a nucleic acid molecule encoding the targeting moiety can comprise a nucleic acid sequence as set forth in any one of SEQ ID NO: 1, 3, 5, 7, and 9 or a nucleic acid sequence having at least 80% homology thereof

In the present application, the start and end of the amino acid sites of the CDR are calculated in Kabat's way.

It is to be understood that the proteins, polypeptides, amino acid sequences and/or nucleic acid sequences involved in the present application also encompasses at least the followings: a variant or a homologue that has the same or similar functions as the proteins or polypeptides, and/or a nucleic acid sequence encoding the variant or homologue that has the same or similar functions as the proteins or polypeptides.

In the present application, the variant can be proteins or polypeptides obtained by substitution, deletion, or addition of one or more amino acids of the proteins and/or polypeptides (e.g., antibodies or fragments thereof that specifically bind to the GPC3 protein, or the low-density lipoprotein receptor-related protein or fragment thereof). For example, the functional variant can comprise a protein or polypeptide with amino acid changes caused by substitution, deletion and/or addition of at least 1 (e.g., 1-30, 1-20, or 1-10, alternatively, e.g., 1, 2, 3, 4, or 5) amino. The functional variant can substantially maintain the biological properties of the protein or polypeptide without change (e.g., substitution, deletion, or addition). For example, the functional variant can maintain at least 60%, 70%, 80%, 90%, or 100% of the biological activities of the protein or the polypeptide (e.g., antigen binding ability, or the biological functions of low-density lipoprotein receptor-related proteins or fragments thereof). For example, the substitution can be a conservative substitution.

In the present application, the homologue can be a protein or polypeptide that bas at least 85% (e.g., at least about 85%, about 90%, about 91%, about 92%, about 93%, about 94%, about 95%, about 96%, about 97%, about 98%, about 99% or higher) sequence homology with the amino acid sequence of an antibody or fragment thereof (e.g., an antibody or fragment thereof that specifically binds to the GPC3 protein, or the low-density lipoprotein receptor-related protein or fragment thereof).

In the present application, the homology generally refers to the similarity, analogy or association between two or more sequences. The “percentage of sequence homology” can be calculated by means of comparing the two sequences to be aligned in a comparison window, and determining the number of sites at which the same nucleic acid bases (e.g., A, T, C, G, I) or the same amino acid residues (e.g., Ala, Pro, Ser, Thr, Gly, Val, Leu, Ile, Phe, Tyr, Trp, Lys, Arg, His, Asp, Glu, Asn, Gln, Cys and Met) are present so as to obtain the number of the matching sites; dividing the number of the matching sites by the total number of sites in the comparison window (i.e., the window size), and multiplying the result by 100 to give the percentage of sequence homology. The alignment to determine the percentage of sequence homology can be achieved in a variety of ways known in the art, e.g., by using a publicly available computer software such as BLAST, BLAST-2, ALIGN, or Megalign (DNASTAR) software. Those skilled in the art can determine the appropriate parameters for sequence alignment, including any algorithm required to achieve maximum alignment within the full-length sequence being compared or within the target sequence region. The homology can also be determined by the following methods: FASTA and BLAST. The description of FASTA algorithm can be found in W. R. Pearson and D. J. Lipman's “Improved Tool for Biological Sequence Comparison”, Proc. Natl. Acad. Sci., 85:2444-2448, 1988; and D. J. Lipman and W. R. Pearson's “Fast and Sensitive Protein Similarity Search”, Science, 227:1435-1441, 1989. For the description of BLAST algorithm, please refer to S. Altschul, W. Gish, W. Miller, E. W. Myers and D. Lipman's “A Basic Local Alignment Search Tool”, Journal of Molecular Biology, 215:403-410, 1990.

In the present application, the CAR can comprise a targeting moiety, a binge region, a transmembrane region, a costimulatory domain, and a signal transduction domain in order from N-terminal to C-terminal.

In the present application, the polynucleotide molecule encoding the CAR can comprise a nucleotide sequence encoding the targeting moiety, a nucleotide sequence encoding the hinge region, a nucleotide sequence encoding the transmembrane region, a nucleotide sequence encoding the transmembrane region, a nucleotide sequence encoding the costimulatory domain, and a nucleotide sequence encoding the signaling domain in order from 5′end to 3′end.

In the present application, the polynucleotide molecule encoding the CAR can further comprise a leader sequence. For example, the nucleic acid molecule encoding the leader sequence can comprise the nucleic acid sequence as set forth in SEQ ID NO: 30 or a nucleic acid sequence having at least 80% homology thereof. The nucleic acid molecule encoding the leader sequence can be located at the 5′end of the nucleotide sequence encoding the targeting moiety.

In the present application, the genetically modified immune cells can express the low-density lipoprotein receptor-related protein or fragments thereof. For example, the genetically modified immune cell can comprise a vector capable of expressing the low-density lipoprotein receptor-related protein or a fragment thereof. The vector can comprise nucleotide molecules encoding the low-density lipoprotein receptor-related protein or fragments thereof. For example, the polynucleotide molecule can comprise a nucleic acid sequence set forth in any one of the following: SEQ ID NO: 21, 23, 25, and 27 or a nucleic acid sequence having at least 80% homology thereof. Alternatively, the vector can be selected from the group consisting of retroviral vectors, lentiviral vectors and/or transposon plasmids.

In the present application, the genetically modified immune cell can express the chimeric antigen receptor (CAR). For example, the genetically modified immune cell can comprise a vector capable of expressing the chimeric antigen receptor (CAR). The vector can comprise a nucleotide molecule encoding the chimeric antigen receptor (CAR). Alternatively, the vector can be selected from the group consisting of retroviral vectors, lentiviral vectors and/or transposon plasmids.

In the present application, the vector capable of expressing the low-density lipoprotein receptor-related protein or fragment thereof and the vector capable of expressing the chimeric antigen receptor (CAR) can be the same or different vectors, as long as the one or more vectors can express the low-density lipoprotein receptor-related protein or fragments thereof and the chimeric antigen receptor (CAR), so that the genetically modified immune cells have both the low-density lipoprotein receptor-related protein or fragment thereof and the chimeric antigen receptor (CAR).

For example, the vector capable of expressing the low-density lipoprotein receptor-related protein or fragment thereof and the vector capable of expressing the chimeric antigen receptor (CAR) can be the same vector. In this vector, the nucleotide molecule encoding the low-density lipoprotein receptor-related protein or fragment thereof and the nucleotide molecule encoding the chimeric antigen receptor (CAR) can be located in the same expression box. For example, the nucleotide molecule encoding the low-density lipoprotein receptor-related protein or a fragment thereof can be located at the 3′end of the nucleotide molecule encoding the chimeric antigen receptor (CAR).

In the present application, the nucleotide molecule encoding the low-density lipoprotein receptor-related protein or fragment thereof can be directly or indirectly linked to the nucleotide molecule encoding the chimeric antigen receptor (CAR). For example, the indirect linking can be a linking via a linker sequence. The 5′end of the linker sequence can be linked to the 3′end of the nucleotide molecule encoding the low-density lipoprotein receptor-related protein or fragment thereof, and the 3′end of the linker sequence can be linked to the 5′end of the nucleotide molecule encoding the chimeric antigen receptor (CAR). In the present application, the linker sequence can comprise the nucleic acid sequence set forth in any one of the following: SEQ ID NO: 19 and 29 or a nucleic acid sequence having at least 80% homology thereof.

In the present application, the low-density lipoprotein receptor-related protein or fragment thereof expressed by the genetically modified immune cell and the chimeric antigen receptor (CAR) can be two independent proteins. That is, the two do not have any interlinking relationship to form any form of di(poly)mer or protein complex. However, the low-density lipoprotein receptor-related protein or fragment thereof expressed by the genetically modified immune cell and the chimeric antigen receptor (CAR) can also be interlinked. For example, in some cases, the two proteins formed by translation are not completely cleaved, so that the low-density lipoprotein receptor-related protein or fragment thereof forms a complex with the chimeric antigen receptor.

### Preparation Method

The present application provides a method for preparing a genetically modified immune cell, including a following step: up-regulating the expression of the low density lipoprotein receptor-associated protein or fragment thereof in the genetically modified immune cell.

The present application further provides a method for preparing the composition, including a following step: up-regulating the expression of the low density lipoprotein receptor-associated protein or fragment thereof in the genetically modified immune cell.

In the present application, the method can further comprise a following step of isolating and obtaining peripheral blood mononuclear cells PBMCs, CD3′T lymphocyte, CD8′T lymphocyte, CD4⁺T lymphocyte or regulatory T cells.

In the present application, the method can comprise a following step: isolating and activating the genetically modified immune cell, wherein the activating comprises application of a T cell medium to the isolated genetically modified immune cell.

In the present application, the T cell medium can be selected from one or more of the following group: DMEM medium, 1640 medium, MEM medium, and X-VIVO medium.

In the present application, the method can further comprise a following step: adding one or more T cell stimulatory factors to the isolated peripheral blood mononuclear cells PBMC.

For example, the T cell stimulatory factors can be selected from the group consisting of B lymphocyte surface antigens antibody, TNF antibody, intracellular polyester, and antibiotics.

Alternatively, the T cell stimulatory factors can be selected from the group consisting of CD3antibody, CD28 antibody, 4-1BB antibody, CD80 antibody, CD86 antibody, PHA, PMA, and ionomycin.

In the present application, the T cell stimulatory factors can comprise CD3 antibody, and the CD3 antibody can be at a concentration of 1-10000 ng/mL. For example, the CD3 antibody can be at a concentration of 1-9000 ng/mL, 1-5000 ng/mL, 1-4000 ng/mL, 1-3000 ng/mL, 1-1000 ng/mL, 1-500 ng/mL, 1-400 ng/mL, 1-300 ng/mL, 1-200 ng/mL, 1-100 ng/mL, 1-50 ng/mL, 1-40 ng/mL, 1-30 ng/mL, 1-20 ng/mL, 1-10 ng/mL or 1-5 ng/mL. In the present application, the T cell stimulatory factors can comprise CD28 antibody, and the CD28 antibody can be at a concentration of 1-10000 ng/ml. For example, the CD28 antibody can be at a concentration of 1-9000 ng/mL, 1-5000 ng/mL, 1-4000 ng/mL, 1-3000 ng/mL, 1-1000 ng/mL, 1-500 ng/mL, 1-400 ng/mL, 1-300 ng/mL, 1-200 ng/mL, 1-100 ng/mL, 1-50 ng/mL, 1-40 ng/mL, 1-30 ng/mL, 1-20 ng/mL, 1-10 ng/mL or 1-5 ng/mL.

In the present application, the method can further comprise adding one or more cytokines into the isolated PBMC. In the present application, the cytokines can comprise interleukins. For example, the interleukins can comprise one or more selected from the group consisting of IL2, IL21, IL7, and IL15.

In the present application, the interleukins can comprise IL2, and the IL2 can be at a concentration of 0.1-10000 U/mL. For example, the IL2 can be at a concentration of 0.1-8000 U/mL, 0.1-6000 U/mL, 0.1-4000 U/mL, 0.1-2000 U/mL, 5-2000 U/mL, 5-1900 U/mL, 5-1800 U/mL, 5-1700 U/mL, 5-1600 U/mL, 5-1500 U/mL, 5-1400 U/mL, 5-1300 U/mL, 5-1200 U/mL, 5-1100 U/mL, 5-1000 U/mL, 5-900 U/mL, 5-800 U/mL, 5-700 U/mL, 5-600 U/mL, 5-500 U/mL, 5-400 U/mL, 5-300 U/mL, 5-200 U/mL, 5-500 U/mL, 5-400 U/mL, 5-300 U/mL, 5-200 U/mL, 5-100 U/mL, 5-50 U/mL, 5-40 U/mL, 5-30 U/mL, 5-20 U/mL or 5-10 U/mL.

In the present application, the interleukins can comprise IL21, and the IL21 can be at a concentration of 0.01-1000 ng/ml. For example, the IL21 can be at a concentration of 0.01-800 ng/mL, 0.01-600 ng/mL, 0.01-400 ng/mL, 0.01-200 ng/mL, 0.01-100 ng/mL, 0.1-100 ng/mL, 0.1-90 ng/mL, 0,1-80 ng/mL, 0.1-70 ng/mL, 0.1-60 ng/mL, 0.1-50 ng/mL, 0.1-40 ng/mL, 0.1-30 ng/mL, 0.1-20 ng/mL, 0.1-10 ng/ml or 0.1-5 ng/mL.

In the present application, the interleukins can comprise IL7, and the IL7 can be at a concentration of 0.01-1000 ng/mL. For example, the IL7 can be at a concentration of 0.01-800 ng/mL, 0.01-600 ng/mL, 0.01-400 ng/mL, 0.01-200 ng/mL, 0.01-100 ng/mL, 0.1-100 ng/mL, 0.1-90 ng/mL, 0.1-80 ng/mL, 0.1-70 ng/mL, 0.1-60 ng/mL, 0.1-50 ng/mL, 0.1-40 ng/mL, 0.1-30 ng/mL, 0.1-20 ng/mL, 0.1-10 ng/ml or 0.1-5 ng/mL.

In the present application, the interleukins can comprise IL IS, and the ILIS can be at a concentration of 0.01-1000 ng/mL. For example, the IL15 can be at a concentration of 0.01-800 ng/mL, 0.01-600 ng/mL, 0.01-400 ng/mL, 0.01-200 ng/mL, 0.01-100 ng/mL, 0.1-100 ng/mL, 0.1-90 ng/mL, 0.1-80 ng/mL, 0.1-70 ng/mL, 0,1-60 ng/mL, 0.1-50 ng/mL, 0.1-40 ng/mL, 0.1-30 ng/mL, 0.1-20 ng/mL, 0.1-10 ng/ml or 0.1-5 ng/mL.

Promoting the Proliferation of Immune Cells, Promoting the Production of Memory Immune Cells, Inhibiting the Differentiation of Immune Cells. Enhancing the Release of Cytokines, and Enhancing the Killing Ability to the Tumor

In one aspect, the present application provides a method for promoting the proliferation of immune cells, including a following step: upregulating the expression of the low density lipoprotein receptor-associated protein or fragment thereof in the immune cells.

In one aspect, the present application provides a method for promoting the production of memory immune cells, including the step of upregulating the expression of the low density lipoprotein receptor-associated protein or fragment thereof in the immune cells, thereby promoting the differentiation of the immune cells to the memory immune cells.

In one aspect, the present application provides a method for inhibiting the differentiation of immune cells, including the step of upregulating the expression of the low density lipoprotein receptor-associated protein or fragment thereof in the immune cells, thereby inhibiting the differentiation of the immune cells to the differentiated immune cells.

In one aspect, the present application provides a method for enhancing the release of cytokines from immune cells, including a following step: upregulating the expression of the low density lipoprotein receptor-associated protein or fragment thereof in the immune cells.

In the present application, the cytokines can comprise interleukin, interferon and/or tumor necrosis factor. In the present application, the cytokines can comprise IL-2, IL4, IL6, IL7, IL10, IL21, TNF-α and/or IFNγ.

In one aspect, the present application provides a method for enhancing the ability of immune cells to kill tumors, including a following step: upregulating the expression of the low density lipoprotein receptor-associated protein or fragment thereof in the immune cells.

In one aspect, the present application provides a method for preventing the tumor recurrence in a subject, including administering immune cells to a subject susceptible to a tumor, wherein the expression of the low density lipoprotein receptor-associated protein or fragment thereof is upregulated in the immune cells.

In one aspect, the present application provides a method for treating tumors in a subject in need thereof, including the step of administering immune cells to the subject, wherein the expression of the low density lipoprotein receptor-associated protein or fragment thereof is upregulated in the immune cells.

In the present application, the tumor can be selected from the group consisting of liver cancer, lung cancer, leukemia, and mesothelioma. In the present application, the method can comprise an in vivo method, and an in vitro method.

In the present application, the up-regulation of the expression can be that the expression of the low-density lipoprotein receptor-related protein or fragment thereof in the immune cells is significantly up-regulated (e.g., by 5% or more, 10% or more, 20% or more, 30% or more, 40% or more, 50% or more, 60% or more, 70% or more, 80% or more, 90% or more, 100% or more) as compared to the control immune cells, wherein the control immune cells are the corresponding immune cells in which the expression of the low-density lipoprotein receptor-related protein or fragment thereof is not substantially up-regulated.

In the present application, the promoting of the production of memory immune cells can be that the number of the memory immune cells produced by the immune cells is substantially up-regulated (e.g., by 5% or more, 10% or more, 20% or more, 30% or more, 40% or more, 50% or more, 60% or more, 70% or more, 80% or more, 90% or more. 100% or more) as compared to the control immune cells, wherein the control immune cells are the corresponding immune cells in which the expression of the low density lipoprotein receptor-associated protein or fragment thereof is not substantially up-regulated.

In the present application, the inhibiting of the differentiation of immune cells can be that the number of the differentiated immune cells produced by the immune cells are substantially down-regulated (e.g., by 5% or more, 10% or more, 20% or more, 30% or more, 40% or more, 50% or more, 60% or more, 70% or more, 80% or more, 90% or more, 100% or more) as compared to the number of the memory immune cells produced by the control immune cells, wherein the control immune cells are the corresponding immune cells in which the expression of the low density lipoprotein receptor-associated protein or fragment thereof is not substantially up-regulated.

In the present application, the enhancing of the release of cytokines from immune cells can be that the number of the cytokines released by the immune cells are substantially up-regulated (e.g., by 5%) or more, 10% or more, 20% or more. 30% or more, 40% or more, 50% or more, 60% or more, 70% or more, 80% or more, 90% or more, 100% or more) as compared to the number of the cytokines released by the control immune cells, wherein the control immune cells are the corresponding immune cells in which the expression of the low density lipoprotein receptor-associated protein or fragment thereof is not substantially up-regulated.

In the present application, the enhancing of the killing ability of the immune cells can be that the ability of the immune cells to kill tumors are substantially increased (e.g., by 5% or more. 10% or more, 20% or more, 30% or more, 40% or more, 50% or more, 60% or more, 70% or more, 80% or more, 90% or more, 100% or more) as compared to the ability of the control immune cells to kill tumors, wherein the control immune cells are the corresponding immune cells in which the expression of the low density lipoprotein receptor-associated protein or fragment thereof is not substantially up-regulated.

In the present application, the prevention of the tumor recurrence in a subject can be that the ability of the immune cells for preventing the tumor recurrence in the subject is substantially increased (e.g., by 5% or more, 10% or more, 20% or more, 30% or more, 40% or more, 50% or more, 60% or more, 70% or more, 80% or more, 90% or more, 100% or more) as compared to the ability of the control immune cells for preventing the tumor recurrence, wherein the control immune cells are the corresponding immune cells in which the expression of the low density lipoprotein receptor-associated protein or fragment thereof is not substantially up-regulated.

In the present application, the treatment of a tumor in a subject in need thereof can be that the therapeutical effect of the immune cells for a tumor is substantially increased (e.g., by 5% or more, 10% or more, 20% or more, 30% or more, 40% or more, 50% or more, 60% or more, 70% or more, 80% or more, 90% or more, 100% or more) as compared to the therapeutical effect of the control immune cells for a tumor, wherein the control immune cells are the corresponding immune cells in which the expression of the low density lipoprotein receptor-associated protein or fragment thereof is not substantially up-regulated.

For example, the corresponding immune cells can comprise lymphocytes. In the present application, the corresponding immune cells can comprise T cells. In the present application, the T cells can comprise memory stem cell-like T cells (TSCM) and/or central memory T cells (TCM). In the present application, the TSCM can be CCR7⁺ and/or CD62L⁺. In the present application, the TSCM can further have one or more properties selected from the group consisting of: CD45RA⁺ or CD45RA⁻, CD45RO⁺ or CD45RO⁻, CD27⁺, CD28⁺, CD127⁺, CD122⁺, CD3⁺, CD4⁺, and CD8⁺. In the present application, the corresponding immune cells can comprise genetically modified immune cells, and the genetically modified immune cells express chimeric antigen receptor (CAR). In the present application, the corresponding immune cells can comprise genetically modified T cells.

In the present application, the level of the low density lipoprotein receptor-associated protein or fragment thereof in the corresponding immune cells can be only the expression level of the expression of the low density lipoprotein receptor-associated protein or fragments thereof in a normal organism, or a substantially un-upregulated expression level as compared with the level of the low density lipoprotein receptor-associated protein or fragment thereof in a normal organism. For example, the substantially un-upregulated level can refer to an up-regulation of at most 4.5%, at most 4%, at most 3%, at most 2%, at most 1%, at most 0).5%, at most 0.3%, at most 0.1%, at most 0.01% or less. In the present application, the corresponding immune cells can be free of a vector including a nucleoside molecule encoding the low density lipoprotein receptor-associated protein or fragment thereof, and/or the corresponding immune cells can be free of a nucleoside molecule encoding the low density lipoprotein receptor-associated protein or fragment thereof.

In the present application, a method for measuring the expression level of the low density lipoprotein receptor-associated protein or fragments thereof and/or the cytokines can comprise quantitative PCR, western blot, and immunohistochemistry.

In the present application, a method for measuring the memory immune cells and/or the differentiated immune cells can comprise flow cytometry cells, immunofluorescence, magnetic bead separation, ELISA, ELISPOT, and quantitative PCR.

In the present application, the evaluation of the therapeutic effect of tumors can utilize an index selected from the group consisting of tumor volume, overall survival (OS), overall remission (DOR), duration of stable disease, disease-free survival (DFS), progression-free survival (PFS), disease control rate (DCR), objective response rate (ORR)) and/or clinical benefit response ratio.

In the present application, the evaluation of the effect of preventing the tumor recurrence in a subject can utilize an index selected from the group consisting of the express level of tumor markers (e.g., tumor-related antigens, tumor-specific genes, and tumor suppressor genes), histological test results (e.g., epithelial hyperplasia, degree of polyp disappearance), imaging test results (e.g., molybdenum target test result) and/or express level of serum markers (e.g., serum free DNA, methylated DNA).

### Composition and Use Thereof

The present application provides a composition including the genetically modified immune cell.

In the present application, the composition can further optionally comprise a pharmaceutically acceptable carrier. The pharmaceutically acceptable carrier can comprise buffers, antioxidants, preservatives, low molecular weight peptides, proteins, hydrophilic polymers, amino acids, sugars, chelating agents, counter ions, metal complexes and/or non-ionic surfactants, etc.

In the present application, the composition can be formulated for oral administration, intravenous administration, intramuscular administration, in situ administration at the tumor site, inhalation, rectal administration, vaginal administration, transdermal administration or administration by subcutaneous depot.

The composition can be used to inhibit tumor growth. For example, the composition of the present application can inhibit or delay the development or progression of the disease, reduce the size of the tumor (or even substantially eliminate the tumor), and/or alleviate and/or stabilize the disease state.

The composition of the present application can comprise a therapeutically effective amount of the antibody or the fragment for antigen-binding thereof. The therapeutically effective amount is a dose required to prevent and/or treat (at least partly treating) a disease or disorder (e.g., cancer) and/or any complications thereof in a subject having or at a risk of developing the disease or disorder.

The present application provides use of the genetically modified cells and/or the composition in manufacture of a drug for treating and/or preventing tumor.

In the present application, the tumor can be selected from the group consisting of liver cancer, lung cancer, leukemia, and mesothelioma.

Without being limited by any theory, the following embodiments are only to illustrate the working methods of the device, method, and system of the present application, and are not used to limit the scope of the present application invention.

### EXAMPLES

### Example 1. Construction of Lentiviral Vector

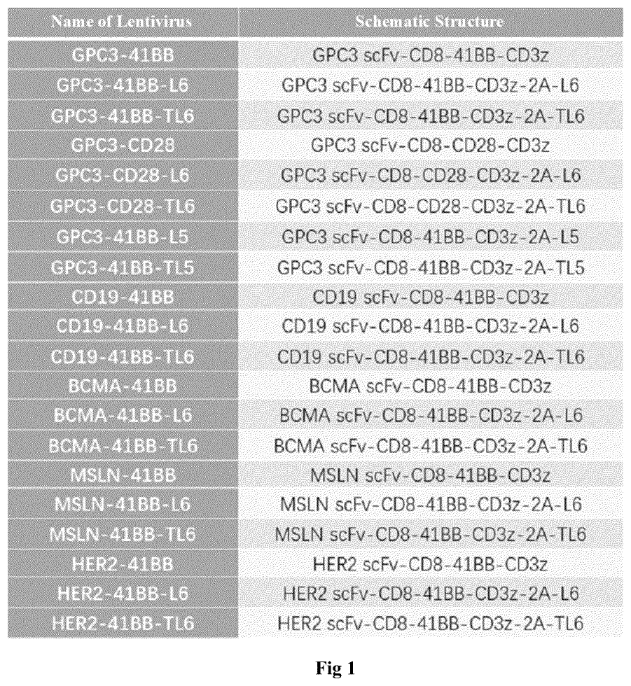

CAR-T takes targeting GPC3, CD19, Mesothelin (MSLN), HER2, BCMA as examples, fragments of the CAR structures are artificially synthesized and constructed into a lentiviral vector (LV100A, System Biosciences), followed by transfection in line with the instructions to obtain lentiviruses (as shown in FIG. 1), that is, GPC3-41BB, GPC3-41BB-L6, GPC3-41BB-TL6,GPC3-CD28, GPC3-CD28-L6, GPC3-CD28-TL6, GPC3-41BB-L5, GPC3-41BB-TL5, BCMA-41BB, BCMA-41BB-L6, BCMA-41BB-TL6, CD19-41BB, CD19-41BB-L6, CD19-41BB-TL6, MSLN-41BB, MSLN-41BB-L6, MSLN-41BB-TL6, HER2-41BB, HER2-41BB-L6,and HER2-41BB-TL6 lentiviruses, respectively.

GPC3-41BB is constructed by successively splicing a leader sequence, GPC3 ScFv, a CD8 hinge region, and a transmembrane region, 41BB, CD3zeta from 5′end to 3′end, of which the nucleotide sequences are as set forth in SEQ ID NO.30, SEQ ID NO.1, SEQ ID NO.11, SEQ ID NO.15, and SEQ ID NO.17.

GPC3-41BB-L6 is constructed by successively splicing a leader sequence, GPC3 ScFv, a CD8 hinge region, and a transmembrane region, 41BB, CD3zeta, 2A, L6 from 5′end to 3′end, of which the nucleotide sequences are as set forth in SEQ ID NO.30, SEQ ID NO.1, SEQ ID NO.11, SEQ ID NO.15, SEQ ID NO.17, SEQ ID NO. 19, and SEQ ID NO.21.

GPC3-41BB-TL6 is constructed by successively splicing a leader sequence, GPC3 ScFv, a CD8 hinge region, and a transmembrane region, 41BB. CD3zeta, 2A, TL6 from 5′end to 3′end, of which the nucleotide sequences are as set forth in SEQ ID NO.30, SEQ ID NO.1, SEQ ID NO.11, SEQ ID NO.15, SEQ ID NO.17, SEQ ID NO.19, and SEQ ID NO.23.

GPC3-41BB-L5 is constructed by successively splicing a leader sequence, GPC3 ScFv, a CD8 hinge region, and a transmembrane region, 41BB, CD3zeta, 2A, L5 from 5′end to 3′end, of which the nucleotide sequences are as set forth in SEQ ID NO.30, SEQ ID NO.1, SEQ ID NO.11, SEQ ID NO.15, SEQ ID NO.17, SEQ ID NO.19, and SEQ ID NO.25.

GPC3-41BB-TL5 is constructed by successively splicing a leader sequence, GPC3 ScFv, a CD8 hinge region, and a transmembrane region, 41BB, CD3zeta, 2A, TL5 from 5′end to 3′end, of which the nucleotide sequences are as set forth in SEQ ID NO.30, SEQ ID NO.1, SEQ ID NO.11, SEQ ID NO.15, SEQ ID NO.17, SEQ ID NO. 19, and SEQ ID NO.27.

GPC3-CD28 is constructed by successively splicing a leader sequence, GPC3 ScFv, a CD8 hinge region, and a transmembrane region, CD28, CD3zeta from 5′end to 3′end, of which the nucleotide sequences are as set forth in SEQ ID NO.30, SEQ ID NO.1, SEQ ID NO.11, SEQ ID NO. 13, and SEQ ID NO.17.

GPC3-CD28-L6 is constructed by successively splicing a leader sequence, GPC3 ScFv, a CD8 hinge region, and a transmembrane region, CD28, CD3zeta, 2A, L6 from 5′end to 3′end, of which the nucleotide sequences are as set forth in SEQ ID NO.30, SEQ ID NO.1, SEQ ID NO.11, SEQ ID NO.13, SEQ ID NO.17, SEQ ID NO. 19, and SEQ ID NO.21.

GPC3-CD28-TL6 is constructed by successively splicing a leader sequence, GPC3 ScFv, a CD8 hinge region, and a transmembrane region, CD28, CD3zeta, 2A, TL6 from 5′end to 3′end, of which the nucleotide sequences are as set forth in SEQ ID NO.30, SEQ ID NO.1, SEQ ID NO.11, SEQ ID NO.13, SEQ ID NO.17, SEQ ID NO. 19, and SEQ ID NO.23.

BCMA-41BB is constructed by successively splicing a leader sequence, BCMAScFv, a CD8 hinge region, and a transmembrane region, 41BB, CD3 zeta from 5′end to 3′end, of which the nucleotide sequences are as set forth in SEQ ID NO.30, SEQ ID NO.5, SEQ ID NO.11, SEQ ID NO.15, and SEQ ID NO.17.

BCMA-41BB-L6 is constructed by successively splicing a leader sequence, BCMAScFv, a CD8 hinge region, and a transmembrane region, 41BB, CD3zeta, 2A, L6 from 5′end to 3′end, of which the nucleotide sequences are as set forth in SEQ ID NO.30, SEQ ID NO.5, SEQ ID NO.11, SEQ ID NO.15. SEQ ID NO.17, SEQ ID NO. 19, and SEQ ID NO.21.

BCMA-41BB-TL6 is constructed by successively splicing a leader sequence, BCMAScFv, a CD8 hinge region, and a transmembrane region, 41BB, CD3zeta, 2A, TL6 from 5′end to 3′end, of which the nucleotide sequences are as set forth in SEQ ID NO.30, SEQ ID NO.5, SEQ ID NO.11, SEQ ID NO.15, SEQ ID NO.17, SEQ ID NO. 19, and SEQ ID NO.23.

CD19-41BB is constructed by successively splicing a leader sequence, CD19ScFv, a CD8 hinge region, and a transmembrane region, 41BB, CD3zeta from 5′end to 3′end, of which the nucleotide sequences are as set forth in SEQ ID NO.30, SEQ ID NO.3, SEQ ID NO.11, SEQ ID NO.15, and SEQ ID NO.17.

CD19-41BB-L6 is constructed by successively splicing a leader sequence, CD19ScFv, a CD8 hinge region, and a transmembrane region, 41BB, CD3zeta, 2A, L6 from 5′end to 3′end, of which the nucleotide sequences are as set forth in SEQ ID NO.30, SEQ ID NO.3, SEQ ID NO.11, SEQ ID NO.15, SEQ ID NO. 17, SEQ ID NO.19, and SEQ ID NO.21.

CD19-41BB-TL6 is constructed by successively splicing a leader sequence, CD19ScFv, a CD8 hinge region, and a transmembrane region, 41BB, CD3zeta, 2A, TL6 from 5′end to 3′end, of which the nucleotide sequences are as set forth in SEQ ID NO.30, SEQ ID NO.3, SEQ ID NO.11, SEQ ID NO.15, SEQ ID NO.17, SEQ ID NO.19, and SEQ ID NO.23.

MSLN-41BB is constructed by successively splicing a leader sequence, MSLNScFv, a CD8 hinge region, and a transmembrane region, 41BB, CD3zeta from 5′end to 3′end, of which the nucleotide sequences are as set forth in SEQ ID NO.30, SEQ ID NO.7, SEQ ID NO.11, SEQ ID NO.15, and SEQ ID NO.17.

MSLN-41BB-L6 is constructed by successively splicing a leader sequence, MSLNScFv, a CD8 hinge region, and a transmembrane region, 41BB, CD3zeta, 2A, L6 from 5′end to 3′end, of which the nucleotide sequences are as set forth in SEQ ID NO.30, SEQ ID NO.7, SEQ ID NO.11, SEQ ID NO.15, SEQ ID NO.17, SEQ ID NO.19, and SEQ ID NO.21.

MSLN-41BB-TL6 is constructed by successively splicing a leader sequence, MSLNScFv, a CD8 hinge region, and a transmembrane region, 41BB, CD3zeta, 2A, TL6 from 5′end to 3′end, of which the nucleotide sequences are as set forth in SEQ ID NO.30, SEQ ID NO.7, SEQ ID NO.11, SEQ ID NO.15, SEQ ID NO.17, SEQ ID NO.19, and SEQ ID NO.23.

HER2-41BB is constructed by successively splicing a leader sequence, HER2ScFv, a CD8 hinge region, and a transmembrane region, 41BB, CD3zeta from 5′end to 3′end, of which the nucleotide sequences are as set forth in SEQ ID NO.30, SEQ ID NO.9, SEQ ID NO.11. SEQ ID NO.15, and SEQ ID NO.17.

HER2-41BB-L6 is constructed by successively splicing a leader sequence, HER2ScFv, a CD8 hinge region, and a transmembrane region, 41BB, CD3zeta, 2A, L6 from 5′end to 3′end, of which the nucleotide sequences are as set forth in SEQ ID NO.30, SEQ ID NO.9, SEQ ID NO.11, SEQ ID NO.15, SEQ ID NO.17, SEQ ID NO.19, and SEQ ID NO.21.

HER2-41BB-TL6 is constructed by successively splicing a leader sequence, HER2 ScFv, a CD8 hinge region, and a transmembrane region, 41BB, CD3zeta, 2A, TL6 from 5′end to 3′end, of which the nucleotide sequences are as set forth in SEQ ID NO.30, SEQ ID NO.9, SEQ ID NO.11, SEQ ID NO.15, SEQ ID NO.17, SEQ ID NO.19, and SEQ ID NO.23.

### Example 2. Infection of T Cells by Lentivirus

Fresh human peripheral blood was separated by ficoll separation solution to obtain more than 1×10⁷ peripheral blood mononuclear lymphocytes (PBMC). The anti-human CD3 and anti-human CD28 antibodies were diluted with PBS to a final concentration of 1 ng/ml. Then, the diluted antibody mixture was added into the cell culture dish, spread evenly on the cell culture dish, and incubated at room temperature for 2 hours. After 2 hours, the antibody mixture was washed once with PBS. Subsequently, the separated PBMCs were re-suspended in a lymphocyte culture medium containing Xvivo15 medium, 5% FBS, 200) U/ml IL2T or Xvivo15 medium, 5% FBS, 20 ng/ml IL21, 10 ng/ml IL7 to a final concentration of 1×10⁶ cells/ml, and then added into a culture dish containing the antibody mixture and cultured at 37° C. and 5% CO₂ for 24 hours to activate T cells.

An amount of T cell culture solution was added synperonic F108 with a final concentration of 1 mg/ml. The mixture was mixed homogeneously, and heated to 37° C. in a water bath to prepare an uninfected reagent. Subsequently, cell culture dishes were prepared for experiments. First, 1 mg/ml anti-human CD3 antibody and 0.5 mg/ml anti-human CD28 antibody were diluted with a PBS buffer at a volume ratio of 1:1000 and mixed homogeneously, and then diluted with retronectin (1 mg/ml) reagent at a volume ratio of 1:40 and mixed homogeneously. The mixture was spread evenly in a cell culture dish and incubated at room temperature for 2 hours. After 2 hours, the mixture was washed with PBS, and the cell culture dish was done.

The activated T cells were diluted with the infection reagent, and the mixture was added into each lentivirus prepared in Example 1 at the ratio of MOI=3 and mixed homogeneously. Then, the mixture was spread evenly in the cell culture dish for lentivirus infection, so as to obtain the T cells expressing GPC3-41BB, GPC3-41BB-L6, GPC3-41BB-TL6, GPC3-CD28, GPC3-CD28-L6, GPC3-CD28-TL6, GPC3-41BB-L5, GPC3-41BB-TL5, BCMA-41BB, BCMA-41BB-L6, BCMA-41BB-TL6, CD19-41BB, CD19-41BB-L6, CD19-41BB-TL6, MSLN-41BB, MSLN-41BB-L6, MSLN-41BB-TL6, HER2-41BB, HER2-41BB-L6 and HER2-41BB-TL6, respectively. After infection, the infected cells were monitored for their densities which were maintained at 1×10⁶ cells/ml. After 14 days, the number of cells could be expanded by 10-100 times.

### Example 3. LRP6, LRP6 Truncated Protein, LRP5 or LRP5 Truncated Protein Promotes the Formation of Memory T Cells

The GPC3-41BB, GPC3-41BB-L6, GPC3-41BB-TL6, GPC3-CD28, GPC3-CD28-L6, GPC3-CD28-TL6, GPC3-41BB-L5, GPC3-41BB-TL5, BCMA-41BB, BCMA-41BB-L6, BCMA-41BB-TL6, CD19-41BB, CD19-41BB-L6, CD19-41BB-TL6, MSLN-41BB, MSLN-41BB-L6, MSLN-41BB-TL6, HER2-41BB, HER2-41BB-L6 and HER2-41BB-TL6 T cells obtained in Example 2 were cultured in vitro in a cell incubator at 37° C. and 5% CO₂ for 9 days or 13 days in total.

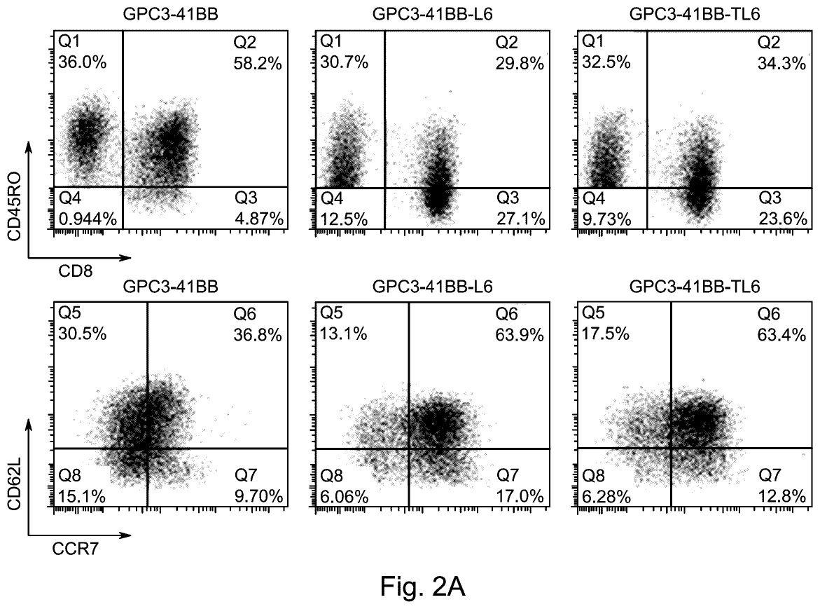

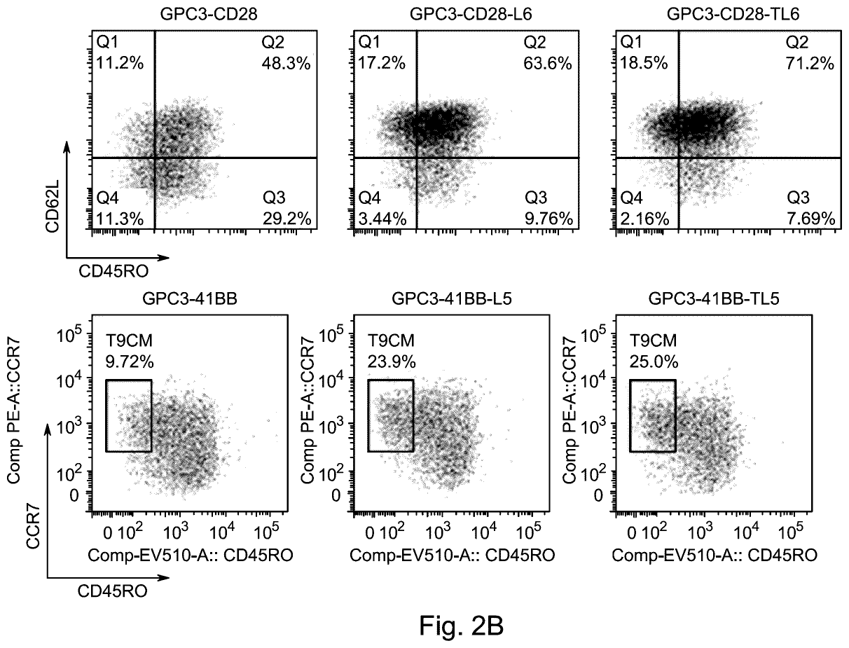

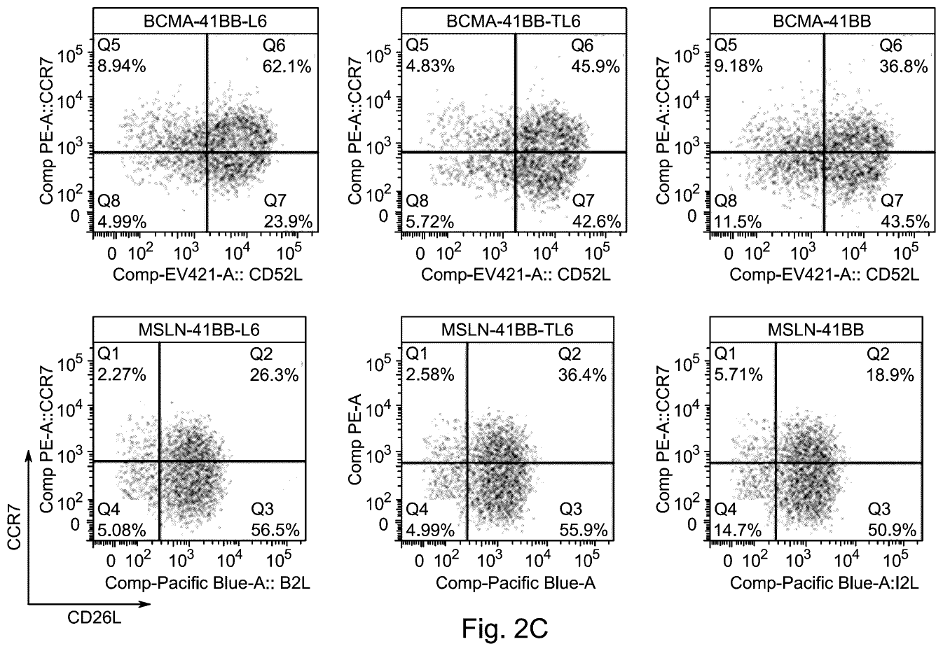

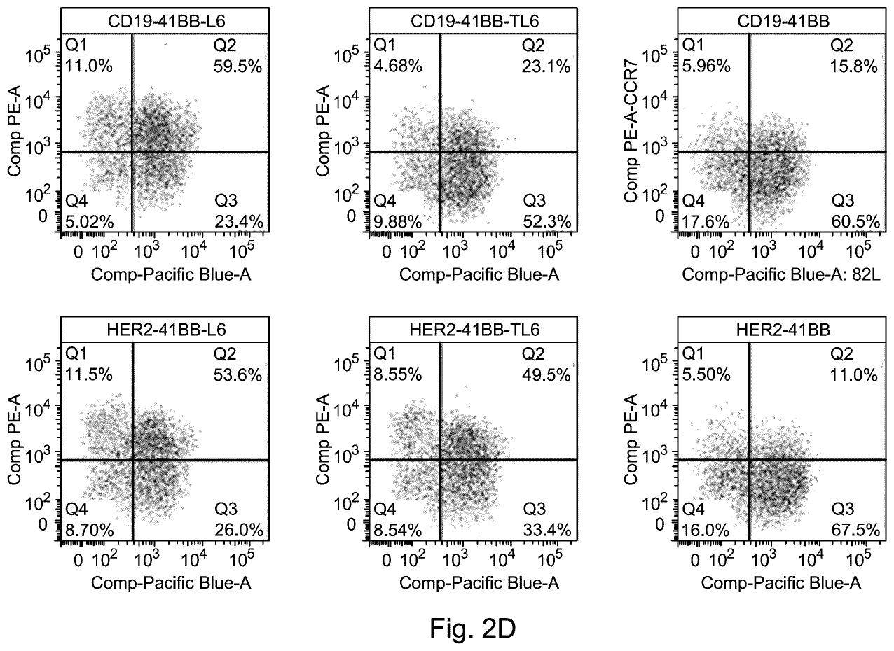

The expression of CD3, CD8, CD45RO, CD45RA, CD62L, CCR7, CD95, CD122, CD127, CD27, CD28 proteins in T cells were detected by BD flow cytometry. Of those, the results of the measured protein expression are shown in FIG. 2A, B, C and D.

The results show that in the T cells expressing GPC3-41BB-L6 or GPC3-41BB-TL6, the proportion of the memory stem cell-like T cells (TSCM) and the central memory T cells (TCM) in the total cells is substantially higher than those in the control group; in the T cells expressing GPC3-CD28-L6 or GPC3-CD28-TL6, the percent of TSCMs and TCMs in the total cells is substantially higher than those in the control group; in the T cells expressing GPC3-41BB-L5 or GPC3-41BB-TL5, the percent of TSCMs and TCMs in the total cells is substantially higher than those in the control group; in the T cells expressing BCMA-41BB-L6 or BCMA-41BB-TL6, the percent of TSCMs and TCMs in the total cells is substantially higher than those in the control group; in the T cells expressing CD19-41BB-L6 or CD19-41BB-TL6, the percent of TSCMs and TCMs in the total cells is substantially higher than those in the control group; in the T cells expressing MSLN-41BB-L6 or MSLN-41BB-TL6, the percent of TSCMs and TCMs in the total cells is substantially higher than those in the control group; and in the T cells expressing HER2-41BB-L6 or HER2-41BB-TL6, the percent of TSCMs and TCMs in the total cells is also substantially higher than those in the control group.

### Example 4 LRP6 or Its Truncated Protein Inhibits the Differentiation of Treg Cells

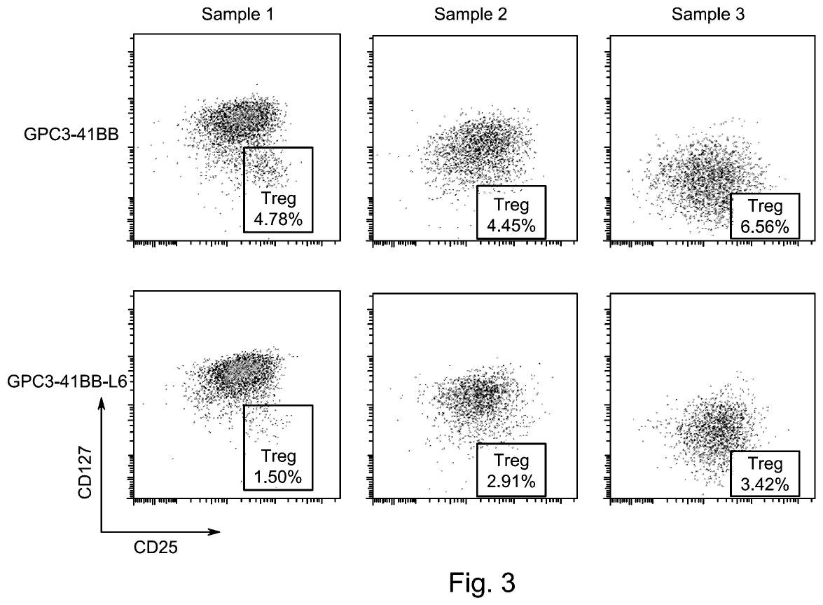

The T cells expressing GPC3-41BB and GPC3-41BB-L6, respectively, as obtained in Example 2 were in vitro cultured in a cell incubator at 37° C., 5% CO₂ for 9 days or 12 days in total. The expression of the CD3, CD4, CD25, CD127, FoxP3 proteins in the T cells were detected by BD flow cytometry, and the measured results of the protein expression are shown in FIG. 3. The results show that in the T cells expressing GPC3-41BB-L6, the percent of the regulatory T cells (Treg) in the CD4″ T cells is significantly lower than that in the control group expressing GPC3-41BB.

### Example 5 LRP6, LRP6 Truncated Protein, LRP5 or LRP5 Truncated Protein Promotes the Amplification of Specific CAR-T Cells Induced by Tumor Antigen

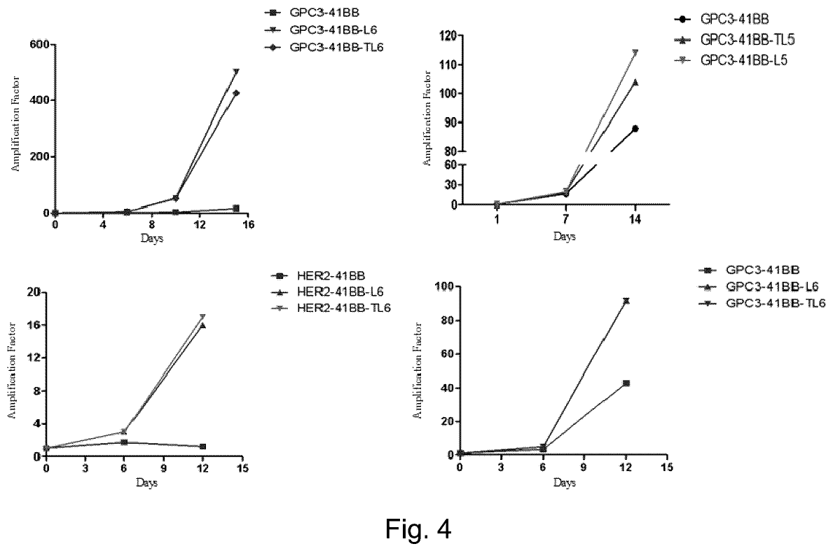

The T cells expressing GPC3-41BB, GPC3-41BB-L6 or GPC3-41BB-TL6 obtained in Example 2 and the radiated Huh7 or HepG2 cells (purchased from the cells library of the Chinese Academy of Sciences) were co-cultured at a ratio of 1:1 in an Xvivo15 medium, supplemented with the radiated Huh7 or HepG2 cells every 5 days for stimulation (3 times in total), and counted with trypan blue every time. The T cells expressing GPC3-41BB, GPC3-41BB-L5 or GPC3-41BB-TL5 and the radiated Huh7 or HepG2 cells (purchased from the cell library of the Chinese Academy of Sciences) were co-cultured with an Xvivo15 medium at a ratio of 1:1, supplemented with the radiated Huh7 or HepG2 cells every 5 days for stimulation (3 times in total), and counted with trypan blue every time. The T cells expressing HER2-41BB, HER2-41BB-L6 or HER2-41BB-TL6 and the radiated SKOV3 cells (purchased from the cells library of the Chinese Academy of Sciences) at a ratio of 1:1 were co-cultured with an Xvivo15 medium, and supplemented with the radiated SKOV3 every 5 days for stimulation (3 times in total), and counted with trypan blue every time. The cell proliferation in each group was shown in FIG. 4. The results show that the amplification factor of the T cells expressing GPC3-41BB-L6 or GPC3-41BB-TL6 T cells is much higher than that in the control group (T cells expressing GPC3-41BB); the amplification factor of the T cells expressing GPC3-41BB-L5 or GPC3-41BB-TL5 is much higher than that of the control group, and the amplification factor of the T cells expressing HER2-41BB-L6 or HER2-41BB-TL6 is also much higher than that of the control group (T cells expressing HER2-41BB).

### Example 6 LRP6 or Its Truncated Protein Promotes the In Vivo Proliferation of CAR-T Cells and the Release of Cytokines

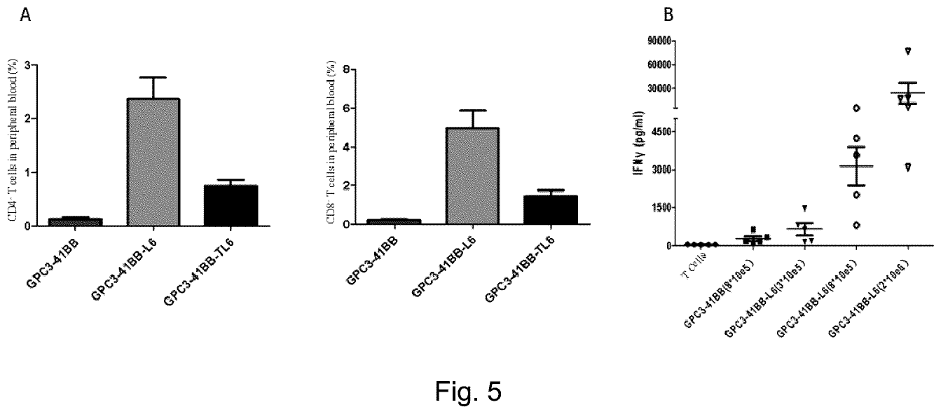

Huh7 cells (1×10⁷ cells/mouse) were subcutaneously inoculated into NSG mice (purchased from Biocyto). After 14 days, the tumor volume of the mice was about 100 mm³. At that time, the mice were divided into 4 groups, i.e., Group T cell, Group GPC3-41BB, Group GPC3-41BB-L6, and Group GPC3-41BB-TL6, and each group contained 6 mice. Then, they were injected through the tail vein with the cells (namely, each group was injected with unmodified T cells, T cells expressing GPC3-41BB, T cells expressing GPC3-41BB-L6, and T cells expressing GPC3-41BB-TL6 at a dose of 3×10⁶ cells/mouse,). On Day 8, a blood sample (50 μl) was taken from the tail of mice. The expression of human CD8 and CD4 proteins was detected by BD flow cytometry in each group. The results are shown in FIG. 5A. The results in FIG. 5A show that the proportion of the cells expressing CD8 and CD4 proteins in the total cells in Group GPC3-41BB-L6 or Group GPC3-41BB-TL6 is much higher than those in other control groups.

Huh7 cells (1×10⁷ cells/mouse) were subcutaneously inoculated into NSG mice (purchased from Biocyto). After 14 days, the tumor volume of the mice was about 100 mm³. At that time, the mice were divided into 5 groups, that is, Group T cell, Group GPC3-41BB, and Group GPC3-41BB-L6, and each group contained 6 mice. Then, the mice were injected via the tail vein with T cells for Group T cell (namely, unmodified T cells at a dose of 2×10⁶ cells/mouse), T cells expressing GPC3-41BB for Group GPC3-41BB (at a dose of 8×10⁵ cells/mouse), and T cells expressing GPC3-41BB-L6 for Group GPC3-41BB-L6 (at a dose of 3×10⁵ cells/mouse, 8×10⁵ cells/mouse or 2×10⁶ cells/mouse). On Day 8, a blood sample (50 μl) was taken from the tail of mice. The expression of human IL-2, IL4, IL6, IL10, TNF-α, and IFNγ cytokines was detected by BD flow cytometry in each group. The results in FIG. 5B show that the expression of IFNγ cytokines in Group GPC3-41BB-L6 is substantially higher than those in other control groups.

### Example 7. LRP6 or Its Truncated Protein Enhances the Anti-Cancer Effect of CAR-T Cells

Huh7 cells (1×10⁷ cells/mouse) were subcutaneously inoculated into NSG mice (purchased from Biocyto). After 14 days, the tumor volume of the mice was about 100 mm³. At that time, the mice were divided into 4 groups, that is, Group T cell, Group GPC3-41BB, and Group GPC3-41BB-L6, respectively, and each group contained 8 mice. Then, the mice were injected via the tail vein with T cells for Group T cells (i.e., unmodified T cells at a dose of 8×10⁵ cells/mouse), T cells expressing GPC3-41BB for Group GPC3-41BB (at a dose of 8×10⁵ cells/mouse), and T cells expressing GPC3-41BB-L6 for Group GPC3-41BB-L6 (at a dose of 3×10⁵ cells/mouse or 8×10⁵ cells/mouse).

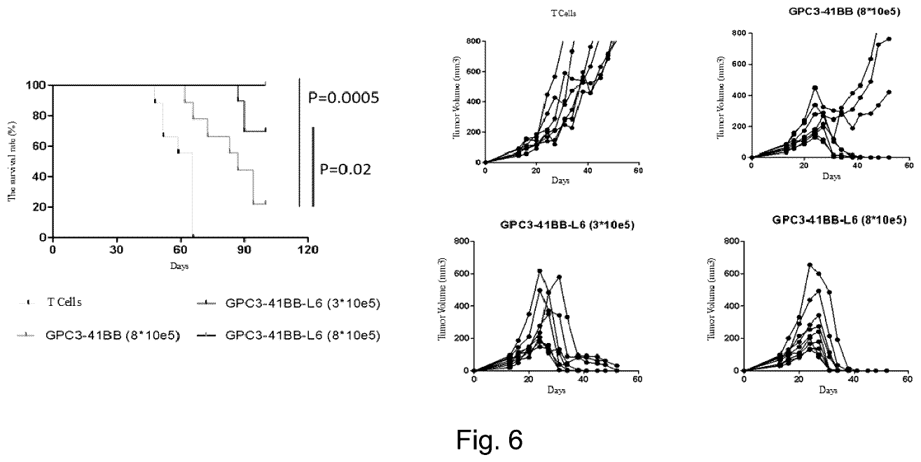

The tumor volume was measured on Monday and Thursday of each week, and the death of the mice was recorded. The results are shown in FIG. 6, and the results show that the tumor-suppressing effect and the survival rate of mice in Group GPC3-41BB-L6 are both significantly higher than those in other control groups, i.e., it is about 3 times that of the control group, Group GPC3-41BB.

### Example 8. LRP6 or Its Truncated Protein Promotes the In Vivo Long-Term Survival of CAR-T Cells and Prevents the Recurrence of Tumor

Huh7 cells (1×10⁷ cells/mouse) were subcutaneously inoculated into NSG mice (purchased from Biocyto). After 14 days, the tumor volume of the mice was about 100 mm³. At that time, the mice were divided into 3 groups, that is, Group T cell, Group GPC3-41BB, and Group GPC3-41BB-L6, and each group contained 9) mice. Then, the mice were injected via the tail vein with T cells for Group T cells (at a dose of 2×10⁶ cells/mouse), T cells expressing GPC3-41BB for Group GPC3-41BB (at a dose of 2×10⁶ cells/mouse), and T cells expressing GPC3-41BB-L6 for Group GPC3-41BB-L6 (at a dose of 2×10⁶ cells/mouse).

After the tumor disappeared for 85 days, the NSG mice were subcutaneously re-inoculated with Huh7 cells (at a dose of 3×10⁷ cells/mouse). On Day 99, 50 μl of blood was sampled from the tail of the mouse, and detected the expression of human CD8, CD4, and CD3 proteins by BD flow cytometry in each group. On Day 120, the mouse bone marrow was sampled, and detected for the expression of human CD8, CD4, and CD3 proteins by BD flow cytometry in each group.

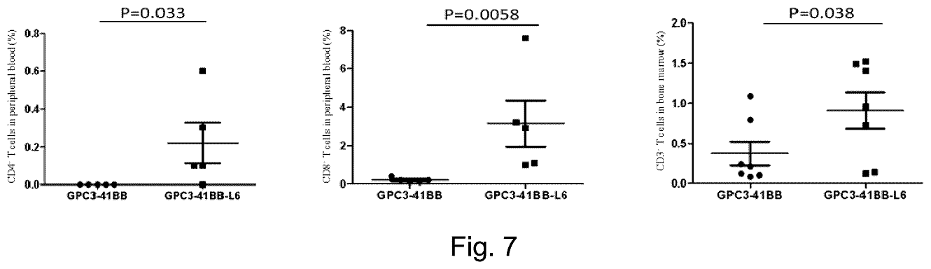

The results are shown in FIG. 7. The results show that the proportion of cells expressing CD8 or CD4 protein in the total cells in the peripheral blood and the proportion of cells expressing CD3 proteins in the total cells in the bone marrow of Group GPC3-41BB-L6 are both much higher GPC3-41BB Group, and the mice in Group GPC3-41BB-L6 cannot form any tumor even if they are inoculated again with tumor cells. It shows that LRP6 or its truncated protein promotes the long-term survival of CAR-T cells in the body and has the characteristics of preventing tumor recurrence.

Example 9 Overexpression of LRP6 and Different Intracellularly Truncated LRP6 (With the C-terminus of the Intracellular Region Truncated) can Promote the Proportion of Memory Cells Tscm of CAR-T Cells and the Cytokine IL-2 Secretion and Proliferation After Activation by Target Cells

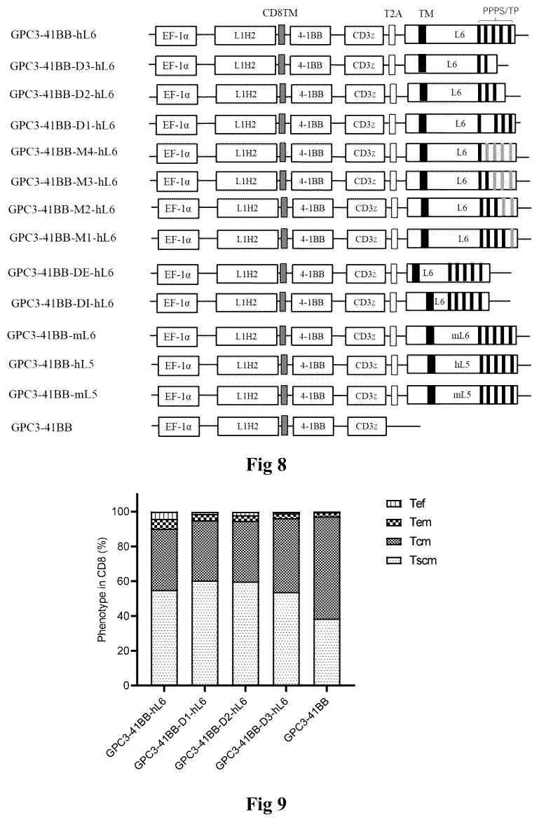

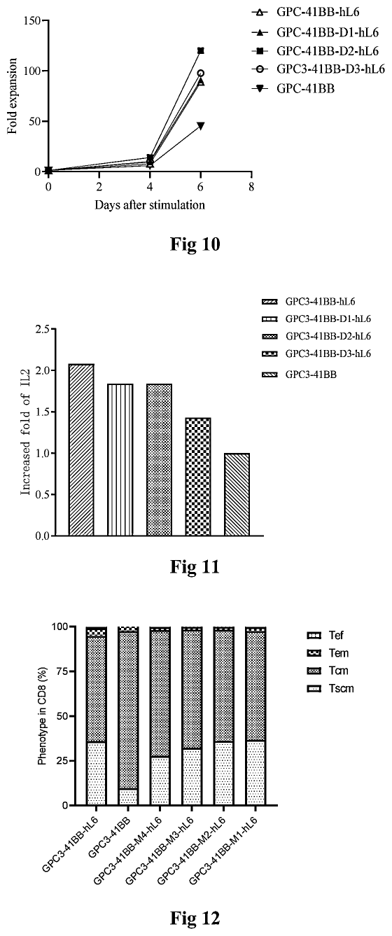

T cells were cultured in a 5% CO₂ cell incubator at 37° C. in vitro for a total of 9 days, which expressed GPC3-41BB-hL6 (the amino acid sequence of hL6 being as set forth in SEQ ID NO: 22), GPC3-41BB-D3-hL6 (the amino acid sequence of D3-hL6 being as set forth in SEQ ID NO: 75), GPC3-41BB-D2-hL6 (the amino acid sequence of D2-hL6 being as set forth in SEQ ID NO: 76), GPC3-41BB-D1-hL6 (the amino acid sequence of D1-hL6 being as set forth in SEQ ID NO: 77) and GPC3-41BB respectively after being transduced by lentivirus with the structure shown in FIG. 8. The expressions of CD3. CD8, CD45RA and CCR7 proteins in T cells were detected by BD flow cytometry, and the detection results of cell memory phenotype are shown in FIG. 9). The harvested CAR-T cells were mixed with target cells huh-7 in a ratio of 1:1, and cultured in a 5% CO₂ cell incubator at 37° C. The proliferated cells were counted by a counter, and the proliferation rates measured are shown in FIG. 10. The culture supernatant collected on Day 2 was subjected to flow cytometry by CBA (cytometric Bead Array), and the detection results of IL2 are shown in FIG. 11.

TABLE 1
Homology of different intracellularly C-terminally truncated L6 and the increased fold of IL-2 secretion of GPC-3 CAR-T
cells expressing different intracellularly C-terminally truncated L6 after stimulation by target cells (donor 1)

| | GPC3-41BB-hL6 | GPC3-41BB-D3-hL6 | GPC3-41BB-D2-hL6 | GPC3-41BB-D1-hL6 | GPC3-41BB |
|---|---|---|---|---|---|
| AA number | 369 | 299 | 334 | 338 | 0 |
| Homology (%) | 100 | 81.0 | 90.5 | 91.6 | 0 |
| Increased fold of IL2 | 2.1 | 1.4 | 1.8 | 1.8 | 1.0 |

Compared with the GPC3-41BB group, for the CAR-T expressing L6 or intracellularly C-terminally truncated L6 (the homology with SEQ ID NO: 22 being 81.0%-91.6%), the proportion of Tscm and the amplification level after stimulation by target cells were improved at various degrees.

Compared with the GPC3-41BB group, for the CAR-T expressing L6 or intracellularly C-terminally truncated L6 (the homology with SEQ ID NO: 22 being 81.0%-91.6%), the IL2 secretion level after stimulation by target cells was increased at various degrees, and the increase was not lower than 40% of the GPC3-41BB control group in the donor of this test.

Example 10 Overexpression of LRP6 and Different Intracellularly C-Terminally Mutated LRP6 can Promote the Proportion of Memory Cells Tscm of CAR-T Cells, and the Cytokine IL2 Secretion and Proliferation After Activation by Target Cells

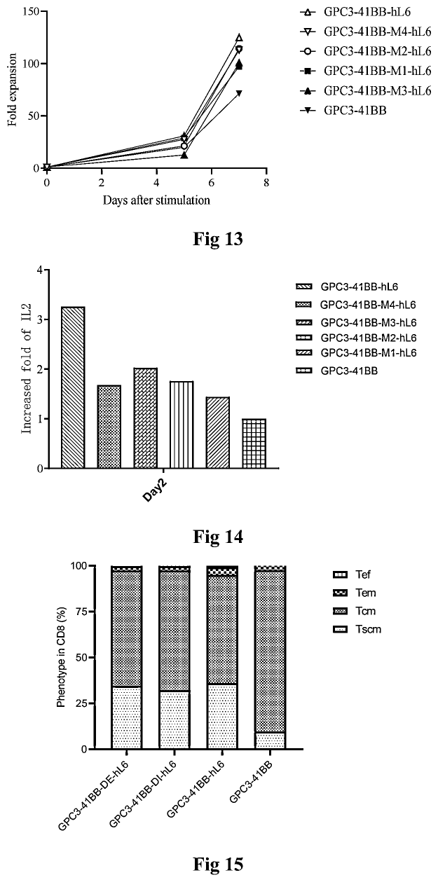

T cells were cultured in a 5% CO₂ cell incubator at 37° C. in vitro for a total of 9 days, which expressed GPC3-41BB-hL6 (the amino acid sequence of hL6 being as set forth in SEQ ID NO: 22), GPC3-41BB-M4-hL6 (the amino acid sequence of M4-hL6 being as set forth in SEQ ID NO: 78), GPC3-41BB-M3-hL6 (the amino acid sequence of M3-hL6 being as set forth in SEQ ID NO: 79), GPC3-41BB-M2-L6 (the amino acid sequence of M2-L6 being as set forth in SEQ ID NO: 80), GPC3-41BB-M1-hL6 (the amino acid sequence of M1-hL6 being as set forth in SEQ ID NO: 81) and GPC3-41BB respectively after being transduced by lentivirus with the structure shown in FIG. 8. The expressions of CD3, CD8, CD45RA and CCR7 proteins in T cells were detected by BD flow cytometry, and the detection results of cell memory phenotype are shown in FIG. 12. The harvested CAR-T cells were mixed with target cells huh-7 in a ratio of 1:1, and cultured in a 5% CO₂ cell incubator at 37° C. The proliferated cells were counted by a counter, and the proliferation rates measured are shown in FIG. 13. The culture supernatant collected on Day 2 was subjected to flow cytometry by CBA (cytometric Bead Array), and the detection results of IL2 are shown in FIG. 14.

TABLE 2
Homology of different intracellularly C-terminally mutated
L6 and the increased fold of IL-2 secretion of GPC-3 CAR-T
cells expressing different intracellularly C-terminally
mutated L6 after stimulation by target cells (donor 2)

| | GPC3- | GPC3- | GPC3- | GPC3- | GPC3- | |
|---|---|---|---|---|---|---|
| | 41BB- | 41BB- | 41BB- | 41BB- | 41BB- | GPC3- |
| | hL6 | M4-hL6 | M3-hL6 | M2-hL6 | M1-hL6 | 41BB |
| AA number | 369 | 364 | 366 | 367 | 368 | 0 |
| Homology (%) | 100 | 98.6 | 99.1 | 99.4 | 99.7 | 0 |
| Increased fold | 3.3 | 1.7 | 2.0 | 1.8 | 1.4 | 1.0 |
| of IL2 | | | | | | |

Compared with the GPC3-41BB group, for the CAR-T expressing L6 or intracellularly C-terminally mutated L6 (the homology with SEQ ID NO: 22 being 98.6%-99.7%), the proportion of Tscm and the amplification level after stimulation by target cells were improved at various degrees.

Compared with the GPC3-41BB group, for the CAR-T expressing L6 or intracellularly C-terminally mutated L6 (the homology with SEQ ID NO: 22 being 98.6%-99.7%), the IL2 secretion level after stimulation by target cells was increased at various degrees, and the increase was not lower than 40% of the GPC3-41BB control group in the donor of this test.

Example 11 Overexpression of LRP6 and Intracellularly and Extracellularly Truncated (Close to the Transmembrane Region) LRP6 can Promote the Proportion of Memory Cells Tscm of CAR-T Cells and the Cytokine IL2 Secretion and Proliferation After Activation by Target Cells

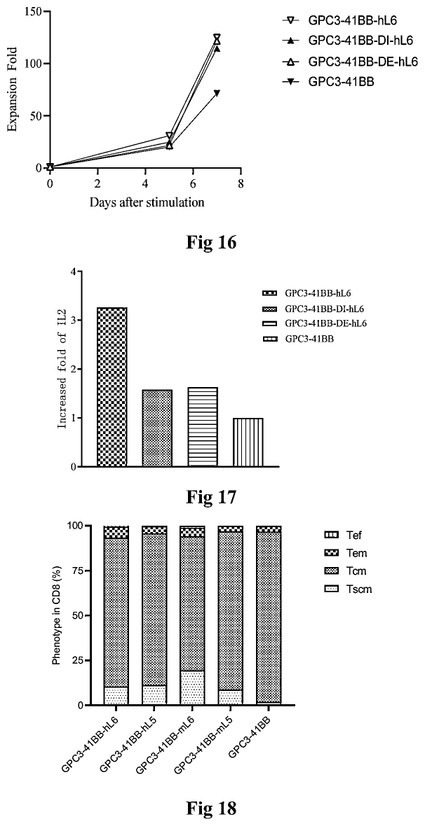

T cells were cultured in a 5% CO₂ cell incubator at 37° C. in vitro for a total of 9 days, which expressed GPC3-41BB-hL6 (the amino acid sequence of hL6 being as set forth in SEQ ID NO: 22), GPC3-41BB-DE-hL6 (the amino acid sequence of DE-hL6 being as set forth in SEQ ID NO: 82), GPC3-41BB-D1-L6 (the amino acid sequence of DI-L6 being as set forth in SEQ ID NO: 83), and GPC3-41BB respectively after being transduced by lentivirus with the structure shown in FIG. 8. The expressions of CD3, CD8, CD45RA and CCR7 proteins in T cells were detected by BD flow cytometry, and the detection results of cell memory phenotype are shown in FIG. 15. The cultured CAR-T cells were mixed with target cells huh-7 in a ratio of 1:1, and cultured in a 5% CO₂ cell incubator at 37° C. The proliferated cells were counted by a counter, and the proliferation rates measured are shown in FIG. 16. The culture supernatant collected on Day 2 was subjected to flow cytometry by CBA (cytometric Bead Array), and the detection results of IL2 are shown in FIG. 17.

TABLE 3
Homology of different intracellularly and extracellularly truncations close to the
transmembrane region and the increased fold of IL2 secretion of GPC-3 CAR-T Cells
expressing different truncated L6 after stimulation by target cells (donor 3)

| | GPC3-41BB-hL6 | GPC3-41BB-DI-hL6 | GPC3-41BB-DE-hL6 | GPC3-41BB |
|---|---|---|---|---|
| AA number | 369 | 249 | 289 | 0 |
| Homology (%) | 100 | 67.5 | 78.3 | 0 |
| Increased fold of IL2 | 3.3 | 1.6 | 1.6 | 1.0 |

Compared with the GPC3-41BB group, for the CAR-T cells expressing L6, DE-hL6 and D1-L6, the Tscm proportion and the amplification level after stimulation by target cells were improved at various degrees.

Compared with the GPC3-41BB group, for the CAR-T cells expressing L6, DE-hL6 and D1-L6, the IL2 secretion level after stimulation by target cells was increased at various degrees, and the increase was not lower than 60% of the GPC3-41BB control group in the donor of this test.

Example 12 Overexpression of LRP6 or LRP5 of Different Species can Promote the Proportion of Memory Cells Tscm of CAR-T Cells and the Cytokine IL2 Secretion and Proliferation After Activation by Target Cells

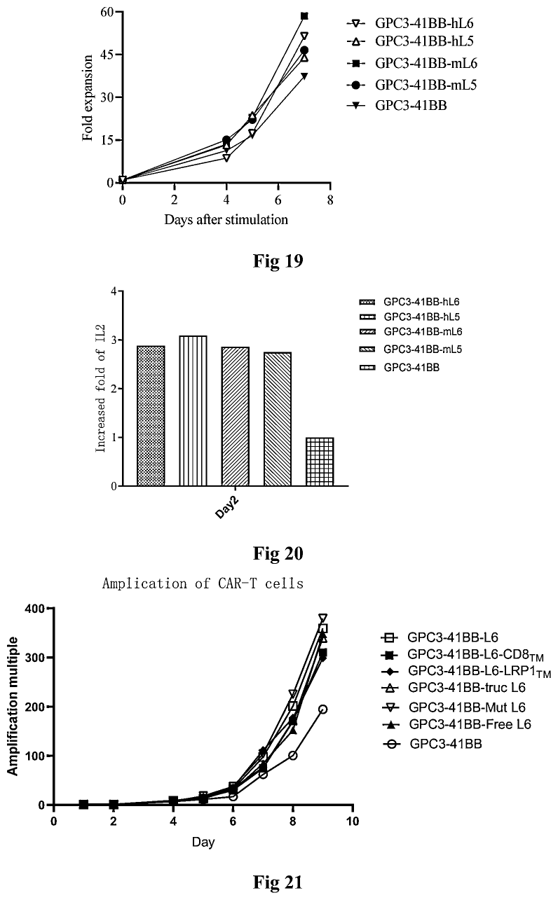

T cells were cultured in a 5% CO₂ cell incubator at 37° C. in vitro for a total of 9 days, which expressed GPC3-41BB-hL6, GPC3-41BB-hL5, GPC3-41BB-mL6 (murine-derived, whose amino acid sequence is as set forth in SEQ ID NO: 84), GPC3-41BB-mL5 (murine-derived, whose amino acid sequence is as set forth in SEQ ID NO: 85), and GPC3-41BB respectively after being transduced by lentivirus with the structure shown in FIG. 8. The expressions of CD3, CD8, CD45RA and CCR7 proteins in T cells were detected by BD flow cytometry, and the detection results of cell memory phenotype are shown in FIG. 18. The cultured CAR-T cells were mixed with target cells huh-7 in a ratio of 1:1, and cultured in a 5% CO₂ cell incubator at 37° C. The proliferated cells were counted by a counter, and the proliferation rates measured are shown in FIG. 19. The culture supernatant collected on Day2 was subjected to flow cytometry by CBA (cytometric Bead Array), and the detection results of IL2 are shown in FIG. 20.

TABLE 4
Homology of murine L5/L6 relative to human L5/L5 and the increased fold of IL-2 secretion of
CAR-T cells expressing L5/L6 of different species after stimulation by target cells (donor 4)

| | GPC3-41BB-hL6 | GPC3-41BB-hL5 | GPC3-41BB-mL6 | GPC3-41BB-mL5 | GPC3-41BB |
|---|---|---|---|---|---|
| AA number | 369 | 361 | 369 | 361 | 0 |
| Homology (%) | 100 | 100 | 98.1 | 92.0 | 0 |
| Increased fold of IL2 | 2.9 | 3.1 | 2.9 | 2.8 | 1.0 |

Compared with the GPC3-41BB group, for the CAR-T expressing L5/L6 of different species (the homology of murine origin to human origin being 92.0-98.1%), the Tscm proportion, amplification level and IL2 secretion level after stimulation by target cells were improved at various degrees, and the effects brought by different species sequences were similar.

### Example 13 LRP6, LRP6 (With Transmembrane Region Replacement) and Intracellularly Truncated/Mutated/Free LRP6 can Promote the Formation of Memory T Cells and Cell Proliferation

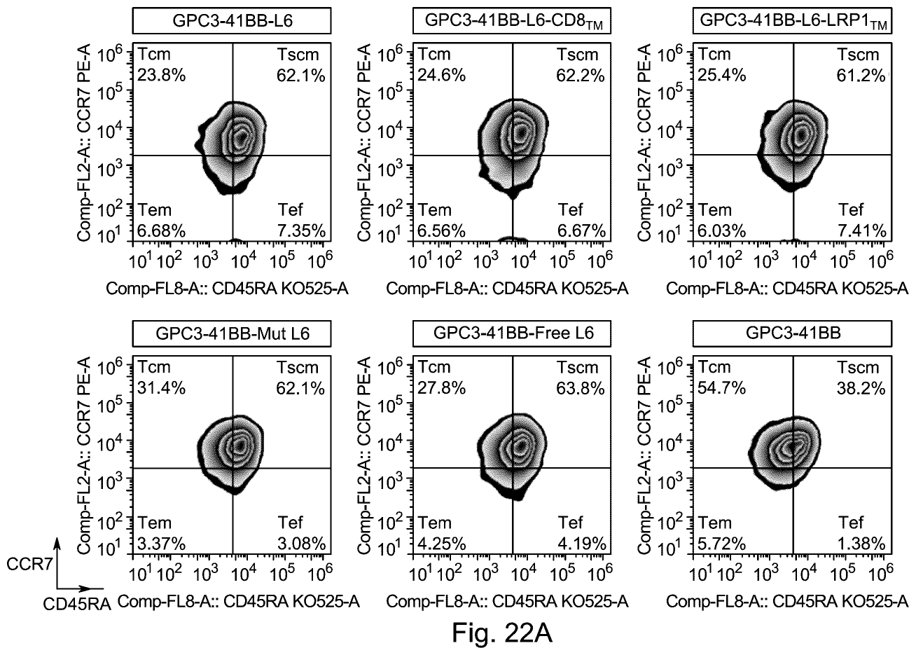

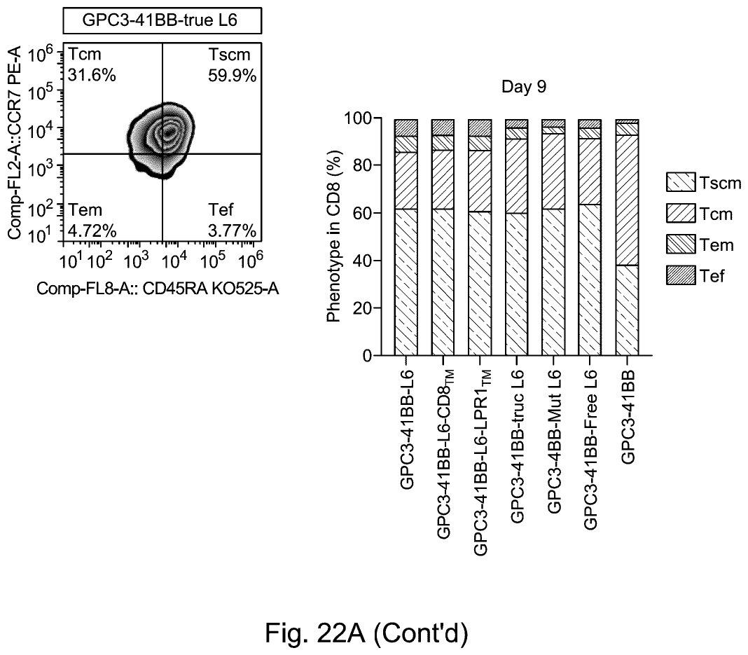

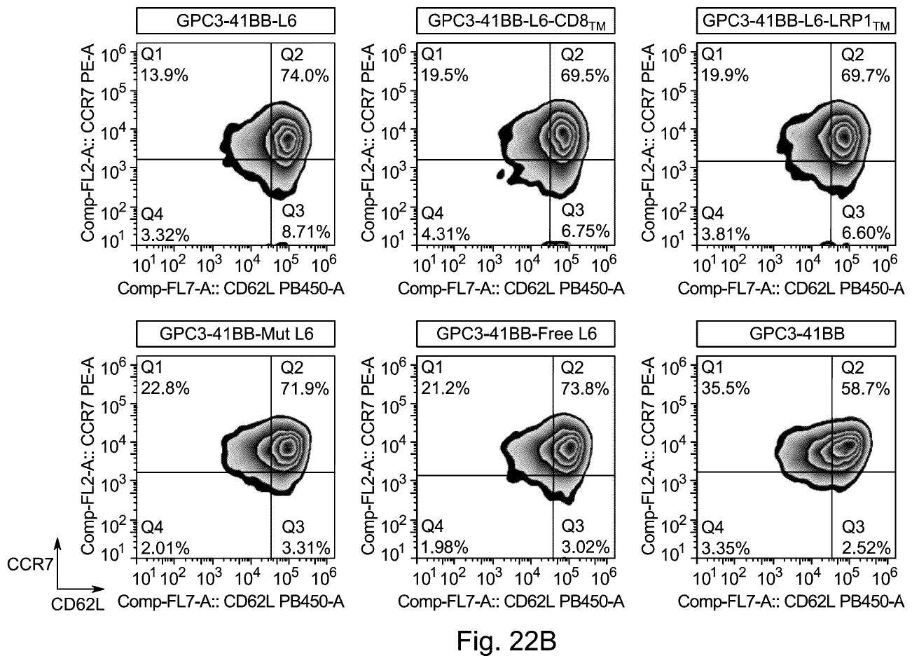

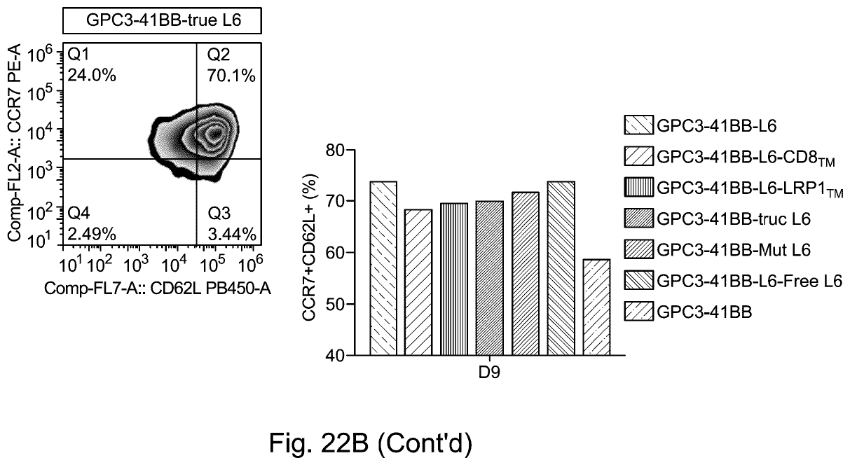

T cells were cultured in a 5% CO₂ cell incubator at 37° C. in vitro for a total of 9 days, which expressed GPC3-41BB-L6-LRP1_{TM}, GPC3-41BB-L6-CD8TM, GPC3-41BB-Free L6,GPC3-41BB-truc L6, GPC3-41BB-Mut L6, GPC3-41BB-L6 and GPC 3-41BB respectively after being transduced by lentivirus with the structure shown in Table 5, wherein the amino acid sequence of L6 is as set forth in SEQ ID NO: 22, the amino acid sequence of Truc L6 is as set forth in SEQ ID NO: 90, the amino acid sequence of Mut L6 is as set forth in SEQ ID NO: 91, the amino acid sequence of Free L6 is as set forth in SEQ ID NO: 89, the amino acid sequence of LRP1-TM is as set forth in SEQ ID NO: 87, the amino acid sequence of CD8-TM is as set forth in SEQ ID NO: 88, and the amino acid sequence of L6 TM is as set forth in SEQ ID NO: 86. The proliferated cells were counted with a counter, and the measured proliferation rates are shown in FIG. 21. The expressions of CD3, CD8, CD45RA, CD62L and CCR7 proteins in T cells were detected by BD flow cytometry, and the measured protein expression results are shown in FIGS. 22A and 22B.

TABLE 5

| Virus name | Schematic structure | Type |
|---|---|---|
| GPC3-41BB | GPC3 scFv-CD8-41BB-CD3z-L6 | LRP6 (with |
| L6-CD8 ™ | (CD8 ™) | transmembrane |
| GPC3-41BB- | GPC3 scFv-CD8-41BB-CD3z-L6 | replacement) |
| L6-LRP1 ™ | (LRP1 ™) | |
| GPC3-41BB- | GPC3 scFv-CD8-41BB-CD3z-Truc L6 | LRP6 intracel- |
| truc L6 | (no PPPS/TP C-terminal sequences) | lularly |
| | | truncated |
| GPC3-41BB- | GPC3 scFv-CD8-41BB-CD3z-Mut L6 | LRP6 intracel- |
| Mut L6 | (C-terminal PPPS/TP being mutated | lularly mutated |
| | into PPPAP) | |
| GPC3-41BB- | GPC3 scFv-CD8-41BB-CD3z-Free L6 | LRP6 intracel- |
| Free L6 | (without extracellular and TM part) | lularly free |

The results showed that for T cells expressing GPC3-41BB-L6, GPC3-41BB-L6-LRP1_{TM}, GPC3-41BB-L6-CD8TM, GPC3-41BB-truc L6, GPC3-41BB-Mut L6, and GPC3-41BB-Free L6 during cell culture, the proliferation rate was higher than that in the control group GPC3-41BB, and there was no significant difference among all groups; in T cells expressing GPC3-41BB-L6, GPC3-41BB-L6-LRP1_{TM}, GPC3-41BB-L6-CD8TM, GPC3-41BB-truc L6, GPC3-41BB-Mut L6, GPC3-41BB-Free L6, and CD8 memory T cell-like T cells (Tscm) were significantly higher than those in the GPC3-41BB control group, and there was no significant difference among all groups; in T cells expressing GPC3-41BB-L6, GPC3-41BB-L6-LRP1_{TM}, GPC3-41BB-L6-CD8_{TM}, GPC3-41BB-truc L6, GPC3-41BB-Mut L6, GPC3-41BB-Free L6, the proportion of CCR7+CD62L+ double-positive cells was higher than that of the GPC3-41BB control group, and there was no significant difference among all groups.

The foregoing detailed description is provided by way of explanation and examples, and is not intended to limit the scope of the appended claims. At present, the various changes of the implementation methods listed in this article are readily apparent to those of ordinary skill in the art, and are encompassed within the scope of the accompanying claims and their equivalences.

We claim:

1. A genetically modified immune cell,
wherein the genetic modification up-regulates the expression of a low density lipoprotein receptor-associated protein or the fragment thereof in the genetically modified immune cell,
wherein the low density lipoprotein receptor-associated protein is low density lipoprotein receptor-associated protein 6 or the low density lipoprotein receptor-associated protein 5, and
wherein the low density lipoprotein receptor-associated protein or the fragment thereof at least comprises the intracellular region of the low density lipoprotein receptor-associated protein.

2. The genetically modified immune cell of claim 1, which has one or more properties selected from the group consisting of:
1) a promoted proliferation of the immune cell;
2) a promoted production of a memory cell;
3) an inhibited differentiation of the immune cell to a regulatory T cell (Treg);
4) an enhanced release of a cytokine from the immune cell; or
5) an enhanced ability of the immune cell to kill a tumor, compared with an unmodified immune cell.

3. The genetically modified immune cell of claim 1, wherein the low density lipoprotein receptor-associated protein or the fragment thereof comprises a human or mouse low density lipoprotein receptor-associated protein.

4. The genetically modified immune cell of claim 1, wherein the low density lipoprotein receptor-associated protein or the fragment thereof further comprises a transmembrane region of the low density lipoprotein receptor-associated protein.

5. The genetically modified immune cell of claim 1, wherein the intracellular region of the low density lipoprotein receptor-associated protein 6 comprises the amino acid sequence from site 24 to site 243 in SEQ NO. 24 or the amino acid sequence having at least 80% homology thereof.

6. The genetically modified immune cell of claim 1, wherein the intracellular region of the low density lipoprotein receptor-associated protein 5 comprises the amino acid sequence from site 24 to site 231 in SEQ NO. 28 or the amino acid sequence having at least 80% homology thereof.

7. The genetically modified immune cell of claim 1, wherein the low density lipoprotein receptor-associated protein or the fragment thereof comprises the amino acid sequence as set forth in any one of SEQ ID NO: 22, 24, 26, and 28, or the amino acid sequence having at least 80% homology thereof.

8. The genetically modified immune cell of claim 1, wherein the low density lipoprotein receptor-associated protein or the fragment thereof comprises the nucleic acid sequence as set forth in any one of SEQ ID NO: 21, 23, 25, and 27 or the nucleic acid sequence having at least 80% homology thereof.

9. The genetically modified immune cell of claim 1, wherein the genetically modified immune cell comprises a genetically modified T cell, NK cell, B cell, dendritic cell, or macrophage.

10. The genetically modified immune cell of claim 1, wherein the genetically modified immune cells express a chimeric antigen receptor (CAR) or a T cell receptor (TCR).

11. A composition, comprising the genetically modified immune cell of claim 1, and optionally pharmaceutically acceptable carrier.

12. A method for:
1) promoting the proliferation of an immune cell;
2) promoting the production of a memory immune cell;
3) inhibiting the differentiation of an immune cell to a regulatory T cell (Treg);
4) enhancing the release of a cytokine from an immune cell; and/or
5) enhancing the ability of an immune cell to kill a tumor,
comprising a following steps:
upregulating the expression of a low density lipoprotein receptor-associated protein or fragment thereof in the immune cell,
wherein the low density lipoprotein receptor-associated protein is low density lipoprotein receptor-associated protein 6 or the low density lipoprotein receptor-associated protein 5, or
wherein the low density lipoprotein receptor-associated protein or the fragment thereof at least comprises the intracellular region of the low density lipoprotein receptor-associated protein.

13. The method of claim 12, wherein the low density lipoprotein receptor-associated protein or the fragment thereof comprises a human or mouse low density lipoprotein receptor-associated protein.

14. The method of claim 12, wherein the low density lipoprotein receptor-associated protein or the fragment thereof further comprises a transmembrane region of the low density lipoprotein receptor-associated protein.

15. The method of claim 12, wherein the intracellular region of the low density lipoprotein receptor-associated protein 6 comprises the amino acid sequence from site 24 to site 243 in SEQ NO. 24 or the amino acid sequence having at least 80% homology thereof.

16. The method of claim 12, wherein the intracellular region of the low density lipoprotein receptor-associated protein 5 comprises the amino acid sequence from site 24 to site 231 in SEQ NO. 28 or the amino acid sequence having at least 80% homology thereof.

17. The method of claim 12, wherein the low density lipoprotein receptor-associated protein or the fragment thereof comprises the amino acid sequence as set forth in any one of SEQ ID NO: 22, 24, 26, and 28, or the amino acid sequence having at least 80% homology thereof.

18. The method of claim 12, wherein the low density lipoprotein receptor-associated protein or the fragment thereof comprises the nucleic acid sequence as set forth in any one of SEQ ID NO: 21, 23, 25, and 27 or the nucleic acid sequence having at least 80% homology thereof.

19. The method of claim 12, wherein the immune cell comprises a genetically modified T cell, NK cell, B cell, dendritic cell, or macrophage.

20. The method of claim 12, wherein the immune cells express a chimeric antigen receptor (CAR) or a T cell receptor (TCR).


SEQUENCE LISTING (91 sequences)

SEQ ID NO: 1 - DNA, 789 bp, synthetic construct
  misc_feature 1..789: note = GPC3 scFv nucleotide
  source 1..789: mol_type = other DNA
atggctctgcctgtgaccgctctgctgctgcctctggctctgctgctgcacgccgcaagacctgatgtcgtgatgacccagtccccactgtccctgccagtgacaccaggagagcctgcatccatctcttgccggagctcccagtctctggtgcacagcaacggcaatacctacctgcactggtatctgcagaagccaggccagagcccccagctgctgatctacaaggtgtccaaccggttctctggagtgccagaccggttcagcggctccggctctggcaccgatttcacactgaagatcagcagggtggaggcagaggacgtgggcgtgtactattgctcccagaatacccacgtgccccctacatttggccagggcaccaagctggagatcaagggaggaggaggcagcggcggaggaggctccggcggcggcggctctcaggtgcagctggtgcagtccggagcagaggtgaagaagcctggagccagcgtgaaggtgtcctgtaaggcctctggctacaccttcacagattatgagatgcactgggtgcggcaggcacctggacagggactggagtggatgggcgccctggacccaaagaccggcgatacagcctactctcagaagtttaagggcagggtgaccctgacagccgacgagagcacctccacagcctatatggagctgtctagcctgcgcagcgaggataccgccgtgtactattgcacccgcttctacagttacacttattgggggcagggcactctggtcacagtctcttca

SEQ ID NO: 2 - protein, 263 aa, synthetic construct
  REGION 1..263: note = GPC3 scFv
MALPVTALLLPLALLLHAARPDVVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSS

SEQ ID NO: 3 - DNA, 726 bp, synthetic construct
  misc_feature 1..726: note = CD19 scFv nucleotide
  source 1..726: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 4 - protein, 242 aa, synthetic construct
  REGION 1..242: note = CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 5 - DNA, 738 bp, synthetic construct
  misc_feature 1..738: note = BCMA scFv nucleotide
  source 1..738: mol_type = other DNA
gacatcgtgctgacccagagccccgcttctttagctgtgtctttaggagagagggccaccatcaattgtcgtgcctccgagtccgtgtccgtgattggcgcccatttaatccactggtaccagcagaaacccggccagccccccaagctgctgatctatttagccagcaatctggagaccggcgtgcccgccagatttagcggaagcggctccggcaccgattttactttaaccatttcctctttacaagctgaggacgccgccatctactactgtttacagagcagaatctttcctcgtaccttcggccaaggtaccaagctggaaatcaagggatccaccagcggctccggaaagcccggttccggagagggcagcacaaagggccaagttcagctcgtgcagagcggctccgagctgaagaagcccggcgcttccgtgaaggtgagctgcaaggccagcggatacaccttcaccgactactccatcaactgggttcgtcaagctcccggtcaaggtctggagtggatgggctggatcaacaccgaaaccagagagcccgcctatgcctacgacttcagaggtcgtttcgtgttctctttagacacaagcgtgagcaccgcctatttacagatcagctctttaaaggccgaggataccgccgtgtactactgcgctcgtgactacagctacgccatggactactggggccaaggtacactggtgaccgtgtccagc

SEQ ID NO: 6 - protein, 246 aa, synthetic construct
  REGION 1..246: note = BCMA scFv
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 7 - DNA, 777 bp, synthetic construct
  misc_feature 1..777: note = MSLN scFv nucleotide
  source 1..777: mol_type = other DNA
caggtgcagctgcaacagagcggccctggcctggtgacaccttcccagaccctcagcctcacctgcgccatctccggcgattccgtgtccagcaatagcgccacctggaactggatcagacagagccctagcagaggcctggagtggctgggcaggacctattataggagcaagtggtacaacgactacgccgtgtccgtcaagtccagaatgagcatcaaccccgacaccagcaagaatcagttcagcctgcagctgaacagcgtgacccccgaggacacagccgtgtattactgcgccaggggcatgatgacctactactacggcatggacgtgtggggccaaggcaccacagtgaccgtgagcagcggcattctgggcagcggaggaggcggatccggcggcggaggcagcggaggaggaggcagccagcctgtgctgacccagtccagctccctgtccgcttcccctggagcctccgcttccctgacctgcaccctgaggtccggcatcaatgtgggcccctacaggatctactggtatcagcagaagcctggcagccccccccagtacctgctgaactacaagagcgacagcgacaagcagcagggcagcggagtgcctagcagattcagcggatccaaggacgcctccgccaatgctggcgtgctgctgatctccggcctgagaagcgaggacgaagccgactactactgcatgatctggcacagcagcgctgccgtgtttggaggcggcacccaactgacagtcctgtcc

SEQ ID NO: 8 - protein, 259 aa, synthetic construct
  REGION 1..259: note = MSLN scFv
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 9 - DNA, 723 bp, synthetic construct
  misc_feature 1..723: note = HER2 scFv nucleotide
  source 1..723: mol_type = other DNA
caggtccagctgcagcagagcggacccgagctcaagaagcccggcgagaccgtgaagatcagctgtaaggccagcggctacccctttaccaactatggaatgaactgggtgaagcaagctcccggccagggcctgaagtggatgggctggatcaacacaagcaccggagaaagcaccttcgctgacgacttcaagggcagatttgacttctccctcgagacctccgccaacaccgcctacctgcagatcaacaatctgaagtccgaggacagcgctacctacttctgcgccagatgggaggtgtaccatggctacgtcccttattggggccaaggcaccaccgtgaccgtgagcagcggaggaggcggttctggaggcggcggcagcggcggcggcggcagcgacattcagctgacccagtcccacaagttcctgagcacctccgtgggcgacagggtgagcattacctgcaaggccagccaggacgtgtacaatgccgtggcctggtaccagcagaagcctggacagagccccaagctgctgatctacagcgcttcctccaggtacaccggcgtcccttccagatttaccggctccggctccggccccgacttcaccttcaccatcagctccgtgcaggccgaagacctggccgtgtacttctgccaacagcacttcaggacccccttcacctttggcagcggaaccaagctggagatcaaa

SEQ ID NO: 10 - protein, 241 aa, synthetic construct
  REGION 1..241: note = HER2 scFv
QVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 11 - DNA, 207 bp, synthetic construct
  misc_feature 1..207: note = CD8 hinge region and transmembrane region nucleotide
  source 1..207: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 12 - protein, 69 aa, synthetic construct
  REGION 1..69: note = CD8 hinge region and transmembrane region
TTTPAPRPPTPAPTIASQPLSLRPEACRPAAGGAVHTRGLDFACDIYIWAPLAGTCGVLLLSLVITLYC

SEQ ID NO: 13 - DNA, 123 bp, synthetic construct
  misc_feature 1..123: note = CD28 intracellular domain nucleotide
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 14 - protein, 41 aa, synthetic construct
  REGION 1..41: note = CD28 intracellular domain
RSKRSRLLHSDYMNMTPRRPGPTRKHYQPYAPPRDFAAYRS

SEQ ID NO: 15 - DNA, 126 bp, synthetic construct
  misc_feature 1..126: note = 41BB intracellular domain nucleotide
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 16 - protein, 42 aa, synthetic construct
  REGION 1..42: note = 41BB intracellular domain
KRGRKKLLYIFKQPFMRPVQTTQEEDGCSCRFPEEEEGGCEL

SEQ ID NO: 17 - DNA, 339 bp, synthetic construct
  misc_feature 1..339: note = CD3zeta nucleotide
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 18 - protein, 112 aa, synthetic construct
  REGION 1..112: note = CD3zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 54 bp, synthetic construct
  misc_feature 1..54: note = 2A nucleotide
  source 1..54: mol_type = other DNA
gagggcagaggaagtcttctaacatgcggtgacgtggaggagaatcccggccct

SEQ ID NO: 20 - protein, 18 aa, synthetic construct
  REGION 1..18: note = 2A
EGRGSLLTCGDVEENPGP

SEQ ID NO: 21 - DNA, 1167 bp, synthetic construct
  misc_feature 1..1167: note = L6 nucleotide
  source 1..1167: mol_type = other DNA
atgggggccgtcctgaggagcctcctggcctgcagcttctgtgtgctcctgagagcggaacctccaacatgttctcctcagcagtttacttgtttcacgggggaaattgactgtatccctgtggcttggcggtgcgatgggtttactgaatgtgaagaccacagtgatgaactcaattgtcctgtatgctcagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctcctga

SEQ ID NO: 22 - protein, 370 aa, synthetic construct
  REGION 1..370: note = L6
GEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 23 - DNA, 804 bp, synthetic construct
  misc_feature 1..804: note = TL6 nucleotide
  source 1..804: mol_type = other DNA
atgggggccgtcctgaggagcctcctggcctgcagcttctgtgtgctcctgagagcgccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctcctga

SEQ ID NO: 24 - protein, 243 aa, synthetic construct
  REGION 1..243: note = TL6
TNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 25 - DNA, 1089 bp, synthetic construct
  misc_feature 1..1089: note = L5 nucleotide
  source 1..1089: mol_type = other DNA
ggagagccgcccacctgctccccggaccagtttgcatgtgccacaggggagatcgactgtatccccggggcctggcgctgtgacggctttcccgagtgcgatgaccagagcgacgaggagggctgccccgtgtgctccgccgcccagttcccctgcgcgcggggtcagtgtgtggacctgcgcctgcgctgcgacggcgaggcagactgtcaggaccgctcagacgaggcggactgtgacgccatctgcctgcccaaccagttccggtgtgcgagcggccagtgtgtcctcatcaaacagcagtgcgactccttccccgactgtatcgacggctccgacgagctcatgtgtgaaatcaccaagccgccctcagacgacagcccggcccacagcagtgccatcgggcccgtcattggcatcatcctctctctcttcgtcatgggtggtgtctattttgtgtgccagcgcgtggtgtgccagcgctatgcgggggccaacgggcccttcccgcacgagtatgtcagcgggaccccgcacgtgcccctcaatttcatagccccgggcggttcccagcatggccccttcacaggcatcgcatgcggaaagtccatgatgagctccgtgagcctgatggggggccggggcggggtgcccctctacgaccggaaccacgtcacaggggcctcgtccagcagctcgtccagcacgaaggccacgctgtacccgccgatcctgaacccgccgccctccccggccacggacccctccctgtacaacatggacatgttctactcttcaaacattccggccactgcgagaccgtacaggccctacatcattcgaggaatggcgcccccgacgacgccctgcagcaccgacgtgtgtgacagcgactacagcgccagccgctggaaggccagcaagtactacctggatttgaactcggactcagacccctatccacccccacccacgccccacagccagtacctgtcggcggaggacagctgcccgccctcgcccgccaccgagaggagctacttccatctcttcccgccccctccgtccccctgcacggactcatcctga

SEQ ID NO: 26 - protein, 362 aa, synthetic construct
  REGION 1..362: note = L5
GEPPTCSPDQFACATGEIDCIPGAWRCDGFPECDDQSDEEGCPVCSAAQFPCARGQCVDLRLRCDGEADCQDRSDEADCDAICLPNQFRCASGQCVLIKQQCDSFPDCIDGSDELMCEITKPPSDDSPAHSSAIGPVIGIILSLFVMGGVYFVCQRVVCQRYAGANGPFPHEYVSGTPHVPLNFIAPGGSQHGPFTGIACGKSMMSSVSLMGGRGGVPLYDRNHVTGASSSSSSSTKATLYPPILNPPPSPATDPSLYNMDMFYSSNIPATARPYRPYIIRGMAPPTTPCSTDVCDSDYSASRWKASKYYLDLNSDSDPYPPPPTPHSQYLSAEDSCPPSPATERSYFHLFPPPPSPCTDSS

SEQ ID NO: 27 - DNA, 696 bp, synthetic construct
  misc_feature 1..696: note = TL5 nucleotide
  source 1..696: mol_type = other DNA
agtgccatcgggcccgtcattggcatcatcctctctctcttcgtcatgggtggtgtctattttgtgtgccagcgcgtggtgtgccagcgctatgcgggggccaacgggcccttcccgcacgagtatgtcagcgggaccccgcacgtgcccctcaatttcatagccccgggcggttcccagcatggccccttcacaggcatcgcatgcggaaagtccatgatgagctccgtgagcctgatggggggccggggcggggtgcccctctacgaccggaaccacgtcacaggggcctcgtccagcagctcgtccagcacgaaggccacgctgtacccgccgatcctgaacccgccgccctccccggccacggacccctccctgtacaacatggacatgttctactcttcaaacattccggccactgcgagaccgtacaggccctacatcattcgaggaatggcgcccccgacgacgccctgcagcaccgacgtgtgtgacagcgactacagcgccagccgctggaaggccagcaagtactacctggatttgaactcggactcagacccctatccacccccacccacgccccacagccagtacctgtcggcggaggacagctgcccgccctcgcccgccaccgagaggagctacttccatctcttcccgccccctccgtccccctgcacggactcatcctga

SEQ ID NO: 28 - protein, 231 aa, synthetic construct
  REGION 1..231: note = TL5
SAIGPVIGIILSLFVMGGVYFVCQRVVCQRYAGANGPFPHEYVSGTPHVPLNFIAPGGSQHGPFTGIACGKSMMSSVSLMGGRGGVPLYDRNHVTGASSSSSSSTKATLYPPILNPPPSPATDPSLYNMDMFYSSNIPATARPYRPYIIRGMAPPTTPCSTDVCDSDYSASRWKASKYYLDLNSDSDPYPPPPTPHSQYLSAEDSCPPSPATERSYFHLFPPPPSPCTDSS

SEQ ID NO: 29 - DNA, 578 bp, synthetic construct
  misc_feature 1..578: note = IRES sequence
  source 1..578: mol_type = other DNA
tccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccaca

SEQ ID NO: 30 - DNA, 63 bp, synthetic construct
  misc_feature 1..63: note = Leading sequence nucleotide
  source 1..63: mol_type = other DNA
atggctctgcctgtgaccgctctgctgctgcctctggctctgctgctgcacgccgcaagacct

SEQ ID NO: 31 - protein, 45 aa, synthetic construct
  REGION 1..45: note = CD8 hinge sequence
TTTPAPRPPTPAPTIASQPLSLRPEACRPAAGGAVHTRGLDFACD

SEQ ID NO: 32 - DNA, 135 bp, synthetic construct
  misc_feature 1..135: note = CD8 hinge sequence nucleotide
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 33 - protein, 24 aa, synthetic construct
  REGION 1..24: note = CD8 transmembrane sequence
IYIWAPLAGTCGVLLLSLVITLYC

SEQ ID NO: 34 - DNA, 72 bp, synthetic construct
  misc_feature 1..72: note = CD8 transmembrane sequence nucleotide
  source 1..72: mol_type = other DNA
atctacatctgggcgcccttggccgggacttgtggggtccttctcctgtcactggttatcaccctttactgc

SEQ ID NO: 35 - protein, 5 aa, synthetic construct
  REGION 1..5: note = GPC3 HCDR1
DYEMH

SEQ ID NO: 36 - protein, 17 aa, synthetic construct
  REGION 1..17: note = GPC3 HCDR2
ALDPKTGDTAYSQKFKG

SEQ ID NO: 37 - protein, 7 aa, synthetic construct
  REGION 1..7: note = GPC3 HCDR3
FYSYTYW

SEQ ID NO: 38 - protein, 16 aa, synthetic construct
RSSQSLVHSNGNTYLH

SEQ ID NO: 39 - protein, 7 aa, synthetic construct
  REGION 1..7: note = GPC3 LCDR2
KVSNRFS

SEQ ID NO: 40 - protein, 9 aa, synthetic construct
  REGION 1..9: note = GPC3 LCDR3
SQNTHVPPT

SEQ ID NO: 41 - protein, 115 aa, synthetic construct
  REGION 1..115: note = GPC3 VH
QVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSS

SEQ ID NO: 42 - protein, 112 aa, synthetic construct
  REGION 1..112: note = GPC3 VL
DVVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIK

SEQ ID NO: 43 - protein, 7 aa, synthetic construct
  REGION 1..7: note = CD19 HCDR1
LPDYGVS

SEQ ID NO: 44 - protein, 16 aa, synthetic construct
  REGION 1..16: note = CD19 HCDR2
VIWGSETTYYNSALKS

SEQ ID NO: 45 - protein, 13 aa, synthetic construct
  REGION 1..13: note = CD19 HCDR3
HYYYGGSYAMDYW

SEQ ID NO: 46 - protein, 11 aa, synthetic construct
  REGION 1..11: note = CD19 LCDR1
RASQDISKYLN

SEQ ID NO: 47 - protein, 7 aa, synthetic construct
  REGION 1..7: note = CD19 LCDR2
HTSRLHS

SEQ ID NO: 48 - protein, 9 aa, synthetic construct
  REGION 1..9: note = CD19 LCDR3
QQGNTLPYT

SEQ ID NO: 49 - protein, 119 aa, synthetic construct
  REGION 1..119: note = CD19 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 50 - protein, 107 aa, synthetic construct
  REGION 1..107: note = CD19 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 51 - protein, 5 aa, synthetic construct
  REGION 1..5: note = BCMA HCDR1
DYSIN

SEQ ID NO: 52 - protein, 17 aa, synthetic construct
  REGION 1..17: note = BCMA HCDR2
WINTETREPAYAYDFRG

SEQ ID NO: 53 - protein, 9 aa, synthetic construct
  REGION 1..9: note = BCMA HCDR3
DYSYAMDYW

SEQ ID NO: 54 - protein, 15 aa, synthetic construct
  REGION 1..15: note = BCMA LCDR1
RASESVSVIGAHLIH

SEQ ID NO: 55 - protein, 7 aa, synthetic construct
  REGION 1..7: note = BCMA LCDR2
LASNLET

SEQ ID NO: 56 - protein, 9 aa, synthetic construct
  REGION 1..9: note = BCMA LCDR3
LQSRIFPRT

SEQ ID NO: 57 - protein, 117 aa, synthetic construct
  REGION 1..117: note = BCMA VH
QVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 58 - protein, 111 aa, synthetic construct
  REGION 1..111: note = BCMA VL
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIK

SEQ ID NO: 59 - protein, 7 aa, synthetic construct
  REGION 1..7: note = MSLN HCDR1
SNSATWN

SEQ ID NO: 60 - protein, 18 aa, synthetic construct
  REGION 1..18: note = MSLN HCDR2
RTYYRSKWYNDYAVSVKS

SEQ ID NO: 61 - protein, 12 aa, synthetic construct
  REGION 1..12: note = MSLN HCDR3
GMMTYYYGMDVW

SEQ ID NO: 62 - protein, 14 aa, synthetic construct
  REGION 1..14: note = MSLN LCDR1
TLRSGINVGPYRIY

SEQ ID NO: 63 - protein, 11 aa, synthetic construct
  REGION 1..11: note = MSLN LCDR2
YKSDSDKQQGS

SEQ ID NO: 64 - protein, 9 aa, synthetic construct
  REGION 1..9: note = MSLN LCDR3
MIWHSSAAV

SEQ ID NO: 65 - protein, 123 aa, synthetic construct
  REGION 1..123: note = MSLN VH
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSS

SEQ ID NO: 66 - protein, 116 aa, synthetic construct
  REGION 1..116: note = MSLN VL
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 67 - protein, 5 aa, synthetic construct
  REGION 1..5: note = HER2 HCDR1
NYGMN

SEQ ID NO: 68 - protein, 17 aa, synthetic construct
  REGION 1..17: note = HER2 HCDR2
WINTSTGESTFADDFKG

SEQ ID NO: 69 - protein, 11 aa, synthetic construct
  REGION 1..11: note = HER2 HCDR3
WEVYHGYVPYW

SEQ ID NO: 70 - protein, 11 aa, synthetic construct
  REGION 1..11: note = HER2 LCDR1
KASQDVYNAVA

SEQ ID NO: 71 - protein, 7 aa, synthetic construct
  REGION 1..7: note = HER2 LCDR2
SASSRYT

SEQ ID NO: 72 - protein, 9 aa, synthetic construct
  REGION 1..9: note = HER2 LCDR3
QQHFRTPFT

SEQ ID NO: 73 - protein, 119 aa, synthetic construct
  REGION 1..119: note = HER2 VH
QVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSS

SEQ ID NO: 74 - protein, 107 aa, synthetic construct
  REGION 1..107: note = HER2 VL
DIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 75 - protein, 300 aa, synthetic construct
  REGION 1..300: note = D3-hL6
GEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAP

SEQ ID NO: 76 - protein, 335 aa, synthetic construct
  REGION 1..335: note = D2-hL6
GEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYL

SEQ ID NO: 77 - protein, 339 aa, synthetic construct
  REGION 1..339: note = D1-hL6
GEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFTDVCDSDYAPSRRMTSVATAKGYTSDLNYDSEPVPPPPTPRSQYLSAEENYESCPPSPYTERSYSHHLYPPPPSPCTDSS

SEQ ID NO: 78 - protein, 370 aa, synthetic construct
  REGION 1..370: note = M4-hL6
GEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPAAPCSTDVCDSDYAPSRRMTSVATAKGYTSDLNYDSEPVPPPPAPRSQYLSAEENYESCPPAPYTERSYSHHLYPPPPAPCTDSS

SEQ ID NO: 79 - protein, 370 aa, synthetic construct
  REGION 1..370: note = M3-hL6
GEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPAPRSQYLSAEENYESCPPAPYTERSYSHHLYPPPPAPCTDSS

SEQ ID NO: 80 - protein, 370 aa, synthetic construct
  REGION 1..370: note = M2-hL6
GEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPAPYTERSYSHHLYPPPPAPCTDSS

SEQ ID NO: 81 - protein, 370 aa, synthetic construct
  REGION 1..370: note = M1-hL6
GEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPAPCTDSS

SEQ ID NO: 82 - protein, 249 aa, synthetic construct
  REGION 1..249: note = DE-hL6
EPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 83 - protein, 290 aa, synthetic construct
  REGION 1..290: note = DI-hL6
GEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 84 - protein, 370 aa, synthetic construct
  REGION 1..370: note = mL6
GEPPTCSPQQFTCFTGDIDCIPVAWRCDGFTECEDHSDELNCPVCSESQFQCASGQCIDGALRCNGDANCQDKSDEKNCEVLCLIDQFRCANGQCVGKHKKCDHSVDCSDRSDELDCYPTEEPAPQATNTVGSVIGVIVTIFVSGTIYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 85 - protein, 362 aa, synthetic construct
  REGION 1..362: note = mL5
GEPPTCSPDQFACTTGEIDCIPGAWRCDGFPECADQSDEEGCPVCSASQFPCARGQCVDLRLRCDGEADCQDRSDEANCDAVCLPNQFRCTSGQCVLIKQQCDSFPDCADGSDELMCEINKPPSDDIPAHSSAIGPVIGIILSLFVMGGVYFVCQRVMCQRYTGASGPFPHEYVGGAPHVPLNFIAPGGSQHGPFPGIPCSKSVMSSMSLVGGRGSVPLYDRNHVTGASSSSSSSTKATLYPPILNPPPSPATDPSLYNVDVFYSSGIPATARPYRPYVIRGMAPPTTPCSTDVCDSDYSISRWKSSKYYLDLNSDSDPYPPPPTPHSQYLSAEDSCPPSPGTERSYCHLFPPPPSPCTDSS

SEQ ID NO: 86 - protein, 23 aa, synthetic construct
  REGION 1..23: note = L6 TM
TNTVGSVIGVIVTIFVSGTVYFI

SEQ ID NO: 87 - protein, 26 aa, synthetic construct
  REGION 1..26: note = LRP1-TM
AVAVLLTILLIVVIGALAIAGFFHYR

SEQ ID NO: 88 - protein, 24 aa, synthetic construct
  REGION 1..24: note = CD8-TM
IYIWAPLAGTCGVLLLSLVITLYC

SEQ ID NO: 89 - protein, 221 aa, synthetic construct
  REGION 1..221: note = Free L6
MCQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 90 - protein, 271 aa, synthetic construct
  REGION 1..271: note = Truc L6
MGAVLRSLLACSFCVLLRAEQKLISEEDLEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILN

SEQ ID NO: 91 - protein, 398 aa, synthetic construct
  REGION 1..398: note = Mut L6
MGAVLRSLLACSFCVLLRAEQKLISEEDLEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPAPATERSHYTMEFGYSSNSPSTHRSYSYRPYSYRHFAPPAAPCSTDVCDSDYAPSRRMTSVATAKGYTSDLNYDSEPVPPPPAPRSQYLSAEENYESCPPAPYTERSYSHHLYPPPPAPCTDSS